(19) United States
(12) Patent Application Publication
(10) Pub. No.: US 2024/0346292 A1
(43) Pub. Date: Oct. 17, 2024

(54) SUPERQUADRATICS NEURAL NETWORK RECONSTRUCTION BY A MAPPING ENGINE OF AN ANATOMICAL STRUCTURE

(71) Applicant: Biosense Webster (Israel) Ltd., Yokneam (IL)

(72) Inventors: Alon BARAM, Yokneam Ilit (IL); Yishayahu Simha GOODMAN, Tel Aviv (IL); Hayit GREENSPAN BECHAR, Tel Aviv (IL); Meir BAR-TAL, Haifa (IL)

(73) Assignee: Biosense Webster (Israel) Ltd., Yokneam (IL)

(21) Appl. No.: 18/632,756

(22) Filed: Apr. 11, 2024

Related U.S. Application Data
(60) Provisional application No. 63/495,665, filed on Apr. 12, 2023.

(51) Int. Cl. G06N 3/0455; G16H 30/40

(52) U.S. Cl. CPC G06N 3/0455; G16H 30/40

(57) ABSTRACT

A method is provided. The method is implemented by a mapping engine. The mapping engine includes processor executable code stored on a memory and executed by a processor. The method includes acquiring catheter trajectories in real-time during an ablation procedure and training a pre-trained neural network based on a dataset and the catheter trajectories to provide a trained neural network. The method includes approximating an atrium shape utilizing the trained neural network and portions of a catheter traversal path and generating a three-dimensional model output from the trained neural network and the atrium shape. The method also includes displaying the three-dimensional model output as an early visualization in the ablation procedure.

### CROSS-REFERENCE TO RELATED APPLICATION

The present invention claims priority based upon U.S. Provisional Appl. No. 63/495,665 filed Apr. 12, 2023, the contents of which are hereby incorporated by reference in their entirety.

### FIELD OF INVENTION

The present invention is related to signal processing. More particularly, the present invention relates to anatomical mapping with respect to signal processing, such a superquadratics neural network reconstruction by a mapping engine of an anatomical structure.

### BACKGROUND

Currently, catheter based radio frequency (RF) ablation for pulmonary vein isolation is a first line of treatment for atrial fibrillation (AF). RF ablation requires a very accurate map of a left atrial sub-endocardial surface, which includes an ostia of the pulmonary veins.

For example, an electroanatomic-mapping (EAM) system tracks the movement of a catheter through the body to create an anatomy surface. To evaluate an efficiency of the EAM system, the EAM system was applied to twenty-five (25) patients undergoing pulmonary vein isolation (PVI) procedures. Fast anatomical mapping (FAM) points were defined as the average of the catheter locations within a one-second respiratory gated window, and the surface was reconstructed using an alpha shape algorithm. The mapping process took an average of nine (9) minutes, with a plus or minus three minute variation (±3). The accuracy of the surface was compared to an MRI scan, resulting in a mean distance of 3.46±0.02 mm and a vein isolation success rate of 96 percent. However, the quality of FAM after 10 minutes is often unsatisfactory, causing physicians to take more than 20 minutes to complete the mapping and may also need to perform manual shape-editing steps.

A solution for a faster and more accurate mapping procedure is greatly needed.

### SUMMARY

According to an exemplary embodiment, a method is provided. The method is implemented by a mapping engine. The mapping engine includes processor executable code stored on a memory and executed by at least one processor. The method includes acquiring one or more catheter trajectories in real-time during an ablation procedure and training a pre-trained neural network based on a dataset and the one or more catheter trajectories to provide a trained neural network. The method includes approximating an atrium shape utilizing the trained neural network and one or more portions of a catheter traversal path and generating a three-dimensional model output from the trained neural network and the atrium shape. The method also includes displaying the three-dimensional model output as an early visualization in the ablation procedure.

According to one or more embodiments, the exemplary method embodiment above can be implemented as an apparatus, a system, and/or a computer program product.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. A more detailed understanding of the techniques described herein may be had from the following description, given by way of example in conjunction with the accompanying drawings, wherein like reference numerals in the figures indicate like elements.

FIG. 1 depicts an example catheter-based electrophysiology mapping and ablation system according to one or more embodiments;

FIG. 2 is a block diagram of an example system for remotely monitoring and communicating patient biometrics according to one or more embodiments;

FIG. 3 is a system diagram of an example of a computing environment in communication with network according to one or more embodiments;

FIG. 4A is a block diagram of an example device in which one or more features of the disclosure can be implemented according to one or more embodiments;

FIG. 4B depicts a graphical depiction of an artificial intelligence system incorporating the example device of FIG. 4A according to one or more embodiments;

FIG. 5 depicts a method performed in the artificial intelligence system of FIG. 4B according to one or more embodiments;

FIG. 6 depicts an exemplary neural network according to one or more embodiments;

FIG. 7 depicts a method according to one or more embodiments; and

FIG. 8 depicts a system according to one or more embodiments;

FIG. 9 depicts transformations according to one or more embodiments;

FIG. 10 depicts models for shape composition guidance according to one or more embodiments;

FIG. 11 depicts a table according to one or more embodiments;

FIG. 12 depicts models according to one or more embodiments;

FIG. 13 depicts models according to one or more embodiments;

FIG. 14 depicts graphs according to one or more embodiments;

FIG. 15 depicts a table according to one or more embodiments;

FIG. 16 depicts reconstructed surfaces according to one or more embodiments;

FIG. 17 depicts reconstructed surfaces according to one or more embodiments;

FIGS. 18A and 18B depict clinical cases according to one or more embodiments;

FIG. 19 depicts a 3D reconstruction according to one or more embodiments;

FIG. 20 depicts 3D reconstructions according to one or more embodiments; and

FIG. 21 depicts reconstructions according to one or more embodiments.

### DETAILED DESCRIPTION

Disclosed herein is a method and/or system for anatomical mapping. More particularly, the method and system relates to a mapping engine that can utilize machine learning/artificial intelligence (ML/AI) to perform anatomical mapping, such as by utilizing a superquadratics neural network reconstruction of an anatomical structure.

The mapping engine (including any AI/ML algorithm therein) is a processor executable code or software that is executable by processing hardware of any technically feasible device such as medical device equipment. For ease of explanation, the mapping engine is described herein with respect to mapping a heart; however, any anatomical structure, body part, organ, or portion thereof can be a target for mapping by the mapping engine described herein. According to an exemplary embodiment, the mapping engine generates maps of an endocardial surface of a left atrium (LA) using a portion of a catheter traversal path (e.g., initial bearings path). The maps can include one or more three-dimensional (3D) models. By way of example, in contrast to conventional mapping techniques, the mapping engine focuses on using a rapidly-acquired catheter path that traverses only anatomical landmarks, with most of the path being in the blood pool and not requiring contact with the surface.

One or more advantages, technical effects, and/or benefits of the mapping engine generating the maps includes providing early visualization, especially for pulmonary veins and important anatomy parts. The early visualization can guide catheters, such as single-shot catheters, with difficulty sampling surfaces. Thus, the mapping engine can reduce cognitive load, simplify procedures, and enable less proficient surgeons to achieve better results. The mapping engine can provide accurate imaging of difficult-to-reach areas of the atrium, such as the ridge between the left superior vein and the left atrial appendage, can improve catheter navigation and visualization of tissue contact during the ablation procedure.

For example, atrial fibrillation (AF) is a common form of cardiac arrhythmia in humans, affecting millions of people worldwide annually. AF is associated with an increased risk of embolic stroke and decreased quality of life. Catheter-based electroanatomic-mapping (EAM) with 3D-guided radiofrequency ablation for pulmonary vein isolation (PVI) is rapidly becoming the first line of treatment for AF. EAM systems record the position and electrical signals of the catheter as the catheter moves inside the chamber. EAM systems use the position and electrical signal data to reconstruct the endocardial surface and approximate the electrical wave that causes chamber contraction. EAM systems generate visualization of the left atrial (“LA”) endocardial surface anatomy, including the pulmonary veins (PVs) and anatomical parts thereof (e.g., LS—left superior, RS—right superior, LI—left inferior, RI—right inferior, LAA—left atrial appendage). Accurate mapping of the LA surface by the EAM systems requires extensive catheter maneuvering, which takes time and requires skilled physicians. The boundary extraction process, referred to as fast anatomical mapping (FAM), involves the catheter touching a large portion of the boundary to extract many surface points. Local electrograms, force indications, and other indices are used in some example to ensure the catheter is in the correct location.

Further, anatomical imaging methods, such as magnetic resonance imaging (MRI) and intra-cardiac ultrasonic catheters, can capture the LA surface by segmenting the LA surface from the acquired image data. The anatomical imaging methods must consider acquisition time, radiation exposure, limited field of view, noise and contrast, and heart shape deformation due to breathing, heartbeat, and pose. An approach to segmentation involves transforming the acquired image into a 3D map indicating the probability that a point belongs to tissue or the blood pool. The segmentation must be smooth and comply with prior knowledge of various anatomical details.

Not only do EAM systems and other anatomical imaging methods fall short of producing sufficiently accurate maps, there are also presently no techniques that reduce mapping time while maintaining anatomical accuracy. Additionally, because physicians can use different workflows, catheters, and imaging guidance methods depending on their preferences and skills, as well as depending on the arrhythmia, available systems and catheters, level of proficiency, and site regulations, EAM systems and other anatomical imaging methods are limited in accommodating the many variations of real-world ablation procedures for AF. The mapping engine herein provides solutions by including a dense encoder-decoder network with a regularization term to reconstruct the shape of the left atrium from partial data, which is derived from catheter maneuvers.

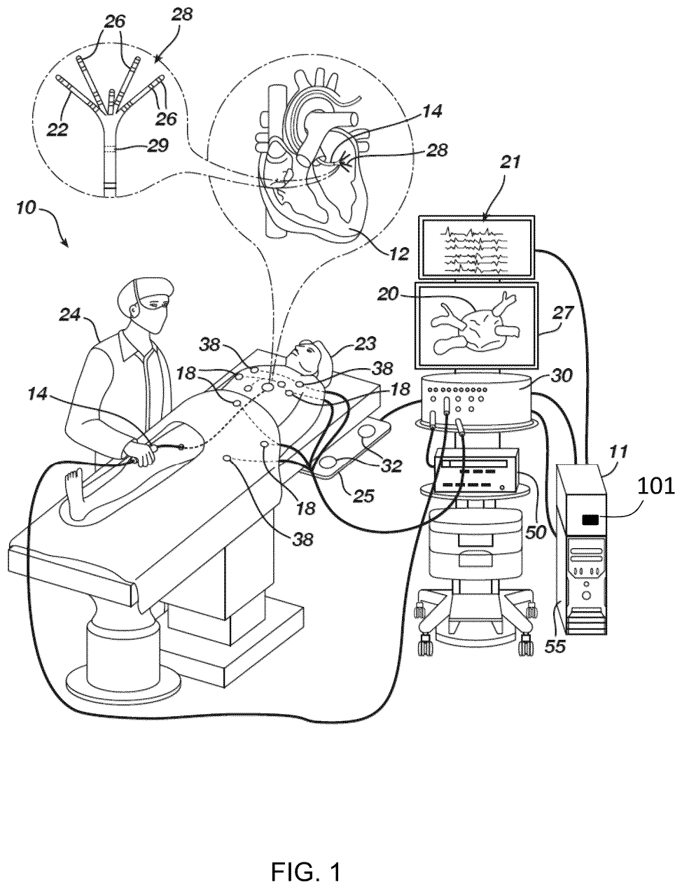

Reference is made to FIG. 1 showing an example system (e.g., medical device equipment and/or catheter-based electrophysiology mapping and ablation system), shown as system 10, in which one or more features of the subject matter herein can be implemented according to one or more embodiments. All or part of the system 100 can be used to collect information (e.g., biometric data and/or a training dataset) and/or used to implement a machine learning and/or an artificial intelligence algorithm (e.g., of a mapping engine) as described herein. The mapping engine 101 (including any AI/ML algorithm therein) is a processor executable code or software that is stored on a memory of the system 10 and that is necessarily rooted in process operations by, and in processing hardware of, the system 10. In various examples, the mapping engine 101 is or includes such software, is or includes hardware (e.g., a fixed function or programmable processor), or is or includes a combination thereof.

The system 10, as illustrated, includes a recorder 11, a heart 12, a catheter 14, a model or anatomical map 20, an electrogram 21, a spline 22, a patient 23, a physician 24 (which is representative of any medical professional, technician, or clinician), a location pad 25, one or more electrodes 26, a display device 27, a distal tip 28, a sensor 29, a coil 32, a patient interface unit (PIU) 30, an electrode skin patches 38, an ablation energy generator 50, and a workstation 55. Note further each element and/or item of the system 10 is representative of one or more of that element and/or that item. The example of the system 10 shown in FIG. 1 can be modified to implement the embodiments disclosed herein. The disclosed embodiments can similarly be applied using other system components and settings. Additionally, the system 10 can include additional components, such as elements for sensing electrical activity, wired or wireless connectors, processing and display devices, or the like.

The system 10 includes multiple catheters 14, which are percutaneously inserted by the physician 24 through the patient's vascular system into a chamber or vascular structure of the heart 12. Typically, a delivery sheath catheter is inserted into the left or right atrium near a desired location in the heart 12. Thereafter, a plurality of catheters can be inserted into the delivery sheath catheter to arrive at the desired location. The plurality of catheters 14 may include catheters dedicated for sensing Intracardiac Electrogram (IEGM) signals, catheters dedicated for ablating, and/or catheters dedicated for both sensing and ablating. The example catheter 14 that is configured for sensing IEGM is illustrated herein. The physician 24 brings the distal tip 28 of the catheter 14 into contact with a heart wall for sensing a target site in the heart 12. For ablation, the physician 24 would similarly bring a distal end of an ablation catheter to a target site for ablating.

The catheter 14 is an exemplary catheter that includes at least one and preferably multiple electrodes 26 optionally distributed over a plurality of splines 22 at the distal tip 28 and configured to sense the IEGM signals. The catheter 14 may additionally include the sensor 29 embedded in or near the distal tip 28 for tracking position and orientation of the distal tip 28. Optionally and preferably, the position sensor 29 is a magnetic based position sensor including three magnetic coils for sensing 3D position and orientation. According to one or more embodiments, shape and parameters of the catheter 14 vary based on whether the catheter 14 is used for diagnostic or ablation purposes, the type of arrhythmia, patient anatomy, and other factors, which affects catheter maneuverability (e.g., an ability to touch without bending the surface and the tracked parts of the catheter 14). The shape and parameters of the catheter 14 also impact the accuracy of anatomical maps. Large spherical single-shot catheters, which can ablate a pulmonary vein within seconds, have become popular but require guidance from fluoroscopy, CT/MRI, or additional mapping catheters. The mapping engine resolves the shortcomings of the catheter 14 as described herein.

The sensor 29 (e.g., a position or a magnetic based position sensor) may be operated together with the location pad 25 including a plurality of magnetic coils 32 configured to generate magnetic fields in a predefined working volume. Real time position of the distal tip 28 of the catheter 14 may be tracked based on magnetic fields generated with the location pad 25 and sensed by the sensor 29. Details of the magnetic based position sensing technology are described in U.S. Pat. Nos. 5,5391,199; 5,443,489; 5,558,091; 6,172,499; 6,239,724; 6,332,089; 6,484,118; 6,618,612; 6,690,963; 6,788,967; 6,892,091.

The system 10 includes one or more electrode patches 38 positioned for skin contact on the patient 23 to establish location reference for the location pad 25 as well as impedance-based tracking of the electrodes 26. For impedance-based tracking, electrical current is directed toward the electrodes 26 and sensed at the patches 38 (e.g., electrode skin patches) so that the location of each electrode can be triangulated via the patches 38. Details of the impedance-based location tracking technology are described in U.S. Pat. Nos. 7,536,218; 7,756,576; 7,848,787; 7,869,865; and 8,456,182, which are incorporated herein by reference.

The recorder 11 displays the electrograms 21 captured with the electrodes 18 (e.g., body surface electrocardiogram (ECG) electrodes) and intracardiac electrograms (IEGM) captured with the electrodes 26 of the catheter 14. The recorder 11 may include pacing capability for pacing the heart rhythm and/or may be electrically connected to a standalone pacer.

The system 10 may include the ablation energy generator 50 that is adapted to conduct ablative energy to the one or more of electrodes 26 at the distal tip 28 of the catheter 14 configured for ablating. Energy produced by the ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

The PIU 30 is an interface configured to establish electrical communication between catheters, electrophysiological equipment, power supply and the workstation 55 for controlling operation of the system 10. Electrophysiological equipment of the system 10 may include for example, multiple catheters 14, the location pad 25, the body surface ECG electrodes 18, the electrode patches 38, the ablation energy generator 50, and the recorder 11. Optionally and preferably, the PIU 30 additionally includes processing capability for implementing real-time computations of location of the catheters and for performing ECG calculations.

The workstation 55 includes memory, processor unit with memory or storage with appropriate operating software loaded therein, and user interface capability (e.g., the memory or storage of the workstation 55 stores the mapping engine 101 and the processor unit of the workstation 55 executes the mapping engine 101). The workstation 55 may provide multiple functions, optionally including (1) modeling the endocardial anatomy in three-dimensions (3D) and rendering the model or anatomical map 20 for display on the display device 27, (2) displaying on the display device 27 activation sequences (or other data) compiled from recorded electrograms 21 in representative visual indicia or imagery superimposed on the rendered anatomical map 20, (3) displaying real-time location and orientation of multiple catheters within the heart chamber, and (5) displaying on the display device 27 sites of interest such as places where ablation energy has been applied. One commercial product embodying elements of the system 10 is available as the CARTO™ 3 System, available from Biosense Webster, Inc., 31A Technology Drive, Irvine, CA 92618.

For instance, the system 10 can be part of a surgical system (e.g., CARTO® system sold by Biosense Webster) that is configured to obtain biometric data (e.g., anatomical and electrical measurements of a patient's organ, such as the heart 12 and as described herein) and perform a cardiac ablation procedure. More particularly, treatments for cardiac conditions such as cardiac arrhythmia often require obtaining a detailed mapping of cardiac tissue, chambers, veins, arteries and/or electrical pathways. For example, a prerequisite for performing a catheter ablation (as described herein) successfully is that the cause of the cardiac arrhythmia is accurately located in a chamber of the heart 12. Such locating may be done via an electrophysiological investigation during which electrical potentials are detected spatially resolved with a mapping catheter (e.g., the catheter 14) introduced into the chamber of the heart 12. This electrophysiological investigation, the so-called electro-anatomical mapping, thus provides 3D mapping data which can be displayed on the display device 27. In many cases, the mapping function and a treatment function (e.g., ablation) are provided by a single catheter or group of catheters such that the mapping catheter also operates as a treatment (e.g., ablation) catheter at the same time.

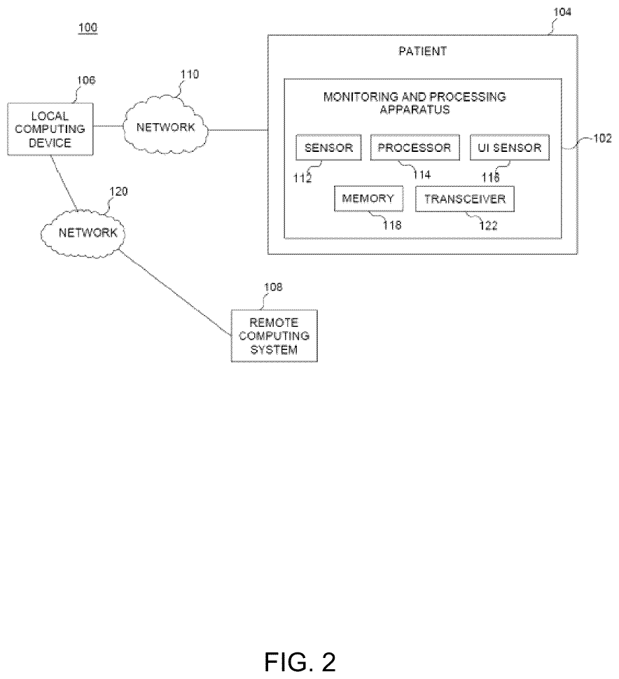

FIG. 2 is a block diagram of an example system 100 for remotely monitoring and communicating patient biometrics (i.e., patient data). In the example illustrated in FIG. 2, the system 100 includes a patient biometric monitoring and processing apparatus 102 associated with a patient 104, a local computing device 106, a remote computing system 108, a first network 110, a patient biometric sensor 112, a processor 114, a user input (UI) sensor 116, a memory 118, a second network 120, and a transmitter-receiver (i.e., transceiver) 122.

According to an embodiment, the patient biometric monitoring and processing apparatus 102 may be an apparatus that is internal to the patient's body (e.g., subcutaneously implantable), such as the catheter 14 of FIG. 1. The patient biometric monitoring and processing apparatus 102 may be inserted into a patient via any applicable manner including orally injecting, surgical insertion via a vein or artery, an endoscopic procedure, or a laparoscopic procedure.

According to an embodiment, the patient biometric monitoring and processing apparatus 102 may be an apparatus that is external to the patient, such as the electrode patches 38 of FIG. 1. For example, as described in more detail below, the patient biometric monitoring and processing apparatus 102 may include an attachable patch (e.g., that attaches to a patient's skin). The monitoring and processing apparatus 102 may also include a catheter with one or more electrodes, a probe, a blood pressure cuff, a weight scale, a bracelet or smart watch biometric tracker, a glucose monitor, a continuous positive airway pressure (CPAP) machine or virtually any device which may provide an input concerning the health or biometrics of the patient.

According to an embodiment, the patient biometric monitoring and processing apparatus 102 may include both components that are internal to the patient and components that are external to the patient.

The single patient biometric monitoring and processing apparatus 102 is shown in FIG. 2. Example systems may, however, may include a plurality of patient biometric monitoring and processing apparatuses. A patient biometric monitoring and processing apparatus may be in communication with one or more other patient biometric monitoring and processing apparatuses. Additionally or alternatively, a patient biometric monitoring and processing apparatus may be in communication with the network 110.

One or more patient biometric monitoring and processing apparatuses 102 may acquire patient biometric data (e.g., electrical signals, blood pressure, temperature, blood glucose level or other biometric data) and receive at least a portion of the patient biometric data representing the acquired patient biometrics and additional formation associated with acquired patient biometrics from one or more other patient biometric monitoring and processing apparatuses 102. The additional information may be, for example, diagnosis information and/or additional information obtained from an additional device such as a wearable device. Each of the patient biometric monitoring and processing apparatus 102 may process data, including its own acquired patient biometrics as well as data received from one or more other patient biometric monitoring and processing apparatuses 102.

Biometric data (e.g., patient biometrics, patient data, or patient biometric data) can include one or more of local activation times (LATs), electrical activity, topology, bipolar mapping, reference activity, ventricle activity, dominant frequency, impedance, or the like. The LAT can be a point in time of a threshold activity corresponding to a local activation, calculated based on a normalized initial starting point. Electrical activity can be any applicable electrical signals that can be measured based on one or more thresholds and can be sensed and/or augmented based on signal to noise ratios and/or other filters. A topology can correspond to the physical structure of a body part or a portion of a body part and can correspond to changes in the physical structure relative to different parts of the body part or relative to different body parts. A dominant frequency can be a frequency or a range of frequency that is prevalent at a portion of a body part and can be different in different portions of the same body part. For example, the dominant frequency of a PV of a heart can be different than the dominant frequency of the right atrium of the same heart. Impedance can be the resistance measurement at a given area of a body part.

Examples of biometric data include, but are not limited to, patient identification data, intracardiac electrocardiogram (IC ECG) data, bipolar intracardiac reference signals, anatomical and electrical measurements, trajectory information, body surface (BS) ECG data, historical data, brain biometrics, blood pressure data, ultrasound signals, radio signals, audio signals, a two- or three-dimensional image data, blood glucose data, and temperature data. The biometrics data can be used, generally, to monitor, diagnosis, and treatment any number of various diseases, such as cardiovascular diseases (e.g., arrhythmias, cardiomyopathy, and coronary artery disease) and autoimmune diseases (e.g., type I and type II diabetes). Note that BS ECG data can include data and signals collected from electrodes on a surface of a patient, IC ECG data can include data and signals collected from electrodes within the patient, and ablation data can include data and signals collected from tissue that has been ablated. Further, BS ECG data, IC ECG data, and ablation data, along with catheter electrode position data, can be derived from one or more procedure recordings.

In FIG. 2, the network 110 is an example of a short-range network (e.g., local area network (LAN), or personal area network (PAN)). Information may be sent, via the network 110, between the patient biometric monitoring and processing apparatus 102 and the local computing device 106 using any one of various short-range wireless communication protocols, such as Bluetooth, Wi-Fi, Zigbee, Z-Wave, near field communications (NFC), ultraband, Zigbee, or infrared (IR).

The network 120 may be a wired network, a wireless network or include one or more wired and wireless networks. For example, the network 120 may be a long-range network (e.g., wide area network (WAN), the internet, or a cellular network,). Information may be sent, via the network 120 using any one of various long-range wireless communication protocols (e.g., TCP/IP, HTTP, 3G, 4G/LTE, or 5G/New Radio).

The patient biometric monitoring and processing apparatus 102 may include the patient biometric sensor 112, the processor 114, the UI sensor 116, the memory 118, and the transceiver 122. The patient biometric monitoring and processing apparatus 102 may continually or periodically monitor, store, process and communicate, via the network 110, any number of various patient biometrics. Examples of patient biometrics include electrical signals (e.g., ECG signals and brain biometrics), blood pressure data, blood glucose data and temperature data. The patient biometrics may be monitored and communicated for treatment across any number of various diseases, such as cardiovascular diseases (e.g., arrhythmias, cardiomyopathy, and coronary artery disease) and autoimmune diseases (e.g., type I and type II diabetes).

The patient biometric sensor 112 may include, for example, one or more sensors configured to sense a type of biometric patient biometrics. For example, the patient biometric sensor 112 may include an electrode configured to acquire electrical signals (e.g., heart signals, brain signals or other bioelectrical signals), a temperature sensor, a blood pressure sensor, a blood glucose sensor, a blood oxygen sensor, a pH sensor, an accelerometer and a microphone.

As described in more detail below, the patient biometric monitoring and processing apparatus 102 may be an ECG monitor for monitoring ECG signals of a heart (e.g., the heart 12). The patient biometric sensor 112 of the ECG monitor may include one or more electrodes for acquiring ECG signals. The ECG signals may be used for treatment of various cardiovascular diseases.

In another example, the patient biometric monitoring and processing apparatus 102 may be a continuous glucose monitor (CGM) for continuously monitoring blood glucose levels of a patient on a continual basis for treatment of various diseases, such as type I and type II diabetes. The CGM may include a subcutaneously disposed electrode, which may monitor blood glucose levels from interstitial fluid of the patient. The CGM may be, for example, a component of a closed-loop system in which the blood glucose data is sent to an insulin pump for calculated delivery of insulin without user intervention.

The transceiver 122 may include a separate transmitter and receiver. Alternatively, the transceiver 122 may include a transmitter and receiver integrated into a single device.

The processor 114 may be configured to store patient data, such as patient biometric data in the memory 118 acquired by the patient biometric sensor 112, and communicate the patient data, across the network 110, via a transmitter of the transceiver 122. Data from one or more other patient biometric monitoring and processing apparatus 102 may also be received by a receiver of the transceiver 122, as described in more detail below.

According to an embodiment, the patient biometric monitoring and processing apparatus 102 includes UI sensor 116 which may be, for example, a piezoelectric sensor or a capacitive sensor configured to receive a user input, such as a tapping or touching. For example, the UI sensor 116 may be controlled to implement a capacitive coupling, in response to tapping or touching a surface of the patient biometric monitoring and processing apparatus 102 by the patient 104. Gesture recognition may be implemented via any one of various capacitive types, such as resistive capacitive, surface capacitive, projected capacitive, surface acoustic wave, piezoelectric and infra-red touching. Capacitive sensors may be disposed at a small area or over a length of the surface such that the tapping or touching of the surface activates the monitoring device.

As described in more detail below, the processor 114 may be configured to respond selectively to different tapping patterns of the capacitive sensor (e.g., a single tap or a double tap), which may be the UI sensor 116, such that different tasks of the patch (e.g., acquisition, storing, or transmission of data) may be activated based on the detected pattern. In some embodiments, audible feedback may be given to the user from the patient biometric monitoring and processing apparatus 102 when a gesture is detected.

The local computing device 106 of the system 100 is in communication with the patient biometric monitoring and processing apparatus 102 and may be configured to act as a gateway to the remote computing system 108 through the second network 120. The local computing device 106 may be, for example, a, smart phone, smartwatch, tablet or other portable smart device configured to communicate with other devices via the network 120. Alternatively, the local computing device 106 may be a stationary or standalone device, such as a stationary base station including, for example, modem and/or router capability, a desktop or laptop computer using an executable program to communicate information between the patient biometric monitoring and processing apparatus 102 and the remote computing system 108 via the PC's radio module, or a USB dongle. Patient biometrics may be communicated between the local computing device 106 and the patient biometric monitoring and processing apparatus 102 using a short-range wireless technology standard (e.g., Bluetooth, Wi-Fi, ZigBee, Z-wave and other short-range wireless standards) via the short-range wireless network 110, such as a local area network (LAN) (e.g., a personal area network (PAN)). In some embodiments, the local computing device 106 may also be configured to display the acquired patient electrical signals and information associated with the acquired patient electrical signals, as described in more detail below.

In some embodiments, the remote computing system 108 may be configured to receive at least one of the monitored patient biometrics and information associated with the monitored patient via network 120, which is a long-range network. For example, if the local computing device 106 is a mobile phone, network 120 may be a wireless cellular network, and information may be communicated between the local computing device 106 and the remote computing system 108 via a wireless technology standard, such as any of the wireless technologies mentioned above. As described in more detail below, the remote computing system 108 may be configured to provide (e.g., visually display and/or aurally provide) the at least one of the patient biometrics and the associated information to a healthcare professional (e.g., a physician).

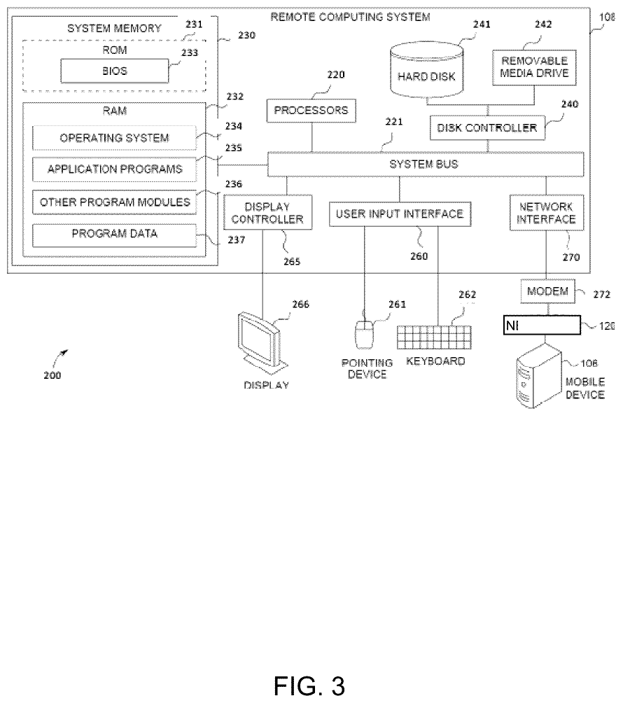

FIG. 3 is a system diagram of an example of a computing environment 200 in communication with network 120. In some instances, the computing environment 200 is incorporated in a public cloud computing platform (such as Amazon Web Services or Microsoft Azure), a hybrid cloud computing platform (such as HP Enterprise OneSphere) or a private cloud computing platform.

As shown in FIG. 3, computing environment 200 includes remote computing system 108 (hereinafter computer system), which is one example of a computing system upon which embodiments described herein may be implemented.

The remote computing system 108 may, via processors 220, which may include one or more processors, perform various functions. The functions may include analyzing monitored patient biometrics and the associated information and, according to physician-determined or algorithm driven thresholds and parameters, providing (e.g., via display 266) alerts, additional information or instructions. As described in more detail below, the remote computing system 108 may be used to provide (e.g., via display 266) healthcare personnel (e.g., a physician) with a dashboard of patient information, such that such information may enable healthcare personnel to identify and prioritize patients having more critical needs than others.

As shown in FIG. 3, the computer system 210 may include a communication mechanism such as a bus 221 or other communication mechanism for communicating information within the computer system 210. The computer system 210 further includes one or more processors 220 coupled with the bus 221 for processing the information. The processors 220 may include one or more CPUs, GPUs, or any other processor known in the art.

The computer system 210 also includes a system memory 230 coupled to the bus 221 for storing information and instructions to be executed by processors 220. The system memory 230 may include computer readable storage media in the form of volatile and/or nonvolatile memory, such as read only system memory (ROM) 231 and/or random-access memory (RAM) 232. The system memory RAM 232 may include other dynamic storage device(s) (e.g., dynamic RAM, static RAM, and synchronous DRAM). The system memory ROM 231 may include other static storage device(s) (e.g., programmable ROM, erasable PROM, and electrically erasable PROM). In addition, the system memory 230 may be used for storing temporary variables or other intermediate information during the execution of instructions by the processors 220. A basic input/output system 233 (BIOS) may contain routines to transfer information between elements within computer system 210, such as during start-up, that may be stored in system memory ROM 231. RAM 232 may comprise data and/or program modules that are immediately accessible to and/or presently being operated on by the processors 220. System memory 230 may additionally include, for example, operating system 234, application programs 235, other program modules 236 and program data 237.

The illustrated computer system 210 also includes a disk controller 240 coupled to the bus 221 to control one or more storage devices for storing information and instructions, such as a magnetic hard disk 241 and a removable media drive 242 (e.g., floppy disk drive, compact disc drive, tape drive, and/or solid state drive). The storage devices may be added to the computer system 210 using an appropriate device interface (e.g., a small computer system interface (SCSI), integrated device electronics (IDE), Universal Serial Bus (USB), or FireWire).

The computer system 210 may also include a display controller 265 coupled to the bus 221 to control a monitor or display 266, such as a cathode ray tube (CRT) or liquid crystal display (LCD), for displaying information to a computer user. The illustrated computer system 210 includes a user input interface 260 and one or more input devices, such as a keyboard 262 and a pointing device 261, for interacting with a computer user and providing information to the processor 220. The pointing device 261, for example, may be a mouse, a trackball, or a pointing stick for communicating direction information and command selections to the processor 220 and for controlling cursor movement on the display 266. The display 266 may provide a touch screen interface that may allow input to supplement or replace the communication of direction information and command selections by the pointing device 261 and/or keyboard 262.

The computer system 210 may perform a portion or each of the functions and methods described herein in response to the processors 220 executing one or more sequences of one or more instructions contained in a memory, such as the system memory 230. Such instructions may be read into the system memory 230 from another computer readable medium, such as a hard disk 241 or a removable media drive 242. The hard disk 241 may contain one or more data stores and data files used by embodiments described herein. Data store contents and data files may be encrypted to improve security. The processors 220 may also be employed in a multi-processing arrangement to execute the one or more sequences of instructions contained in system memory 230. In alternative embodiments, hard-wired circuitry may be used in place of or in combination with software instructions. Thus, embodiments are not limited to any specific combination of hardware circuitry and software.

As stated above, the computer system 210 may include at least one computer readable medium or memory for holding instructions programmed according to embodiments described herein and for containing data structures, tables, records, or other data described herein. The term computer readable medium as used herein refers to any non-transitory, tangible medium that participates in providing instructions to the processor 220 for execution. A computer readable medium may take many forms including, but not limited to, non-volatile media, volatile media, and transmission media. Non-limiting examples of non-volatile media include optical disks, solid state drives, magnetic disks, and magneto-optical disks, such as hard disk 241 or removable media drive 242. Non-limiting examples of volatile media include dynamic memory, such as system memory 230. Non-limiting examples of transmission media include coaxial cables, copper wire, and fiber optics, including the wires that make up the bus 221. Transmission media may also take the form of acoustic or light waves, such as those generated during radio wave and infrared data communications.

The computing environment 200 may further include the computer system 210 operating in a networked environment using logical connections to local computing device 106 and one or more other devices, such as a personal computer (laptop or desktop), mobile devices (e.g., patient mobile devices), a server, a router, a network PC, a peer device or other common network node, and typically includes many or all of the elements described above relative to computer system 210. When used in a networking environment, computer system 210 may include modem 272 for establishing communications over a network 120, such as the Internet. Modem 272 may be connected to system bus 221 via network interface 270, or via another appropriate mechanism.

Network 120, as shown in FIGS. 2 and 3, may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between computer system 610 and other computers (e.g., local computing device 106).

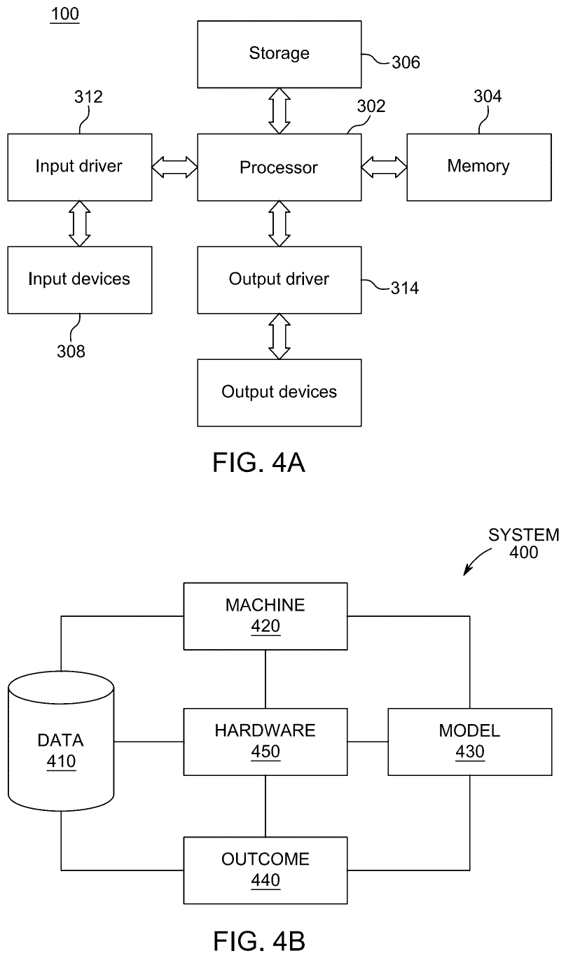

FIG. 4A is a block diagram of an example device 300 in which one or more features of the disclosure can be implemented. The device 300 may be local computing device 106, for example. The device 300 can include, for example, a computer, a gaming device, a handheld device, a set-top box, a television, a mobile phone, or a tablet computer. The device 300 includes a processor 302, a memory 304, a storage device 306, one or more input devices 308, and one or more output devices 310. The device 300 can also optionally include an input driver 312 and an output driver 314. It is understood that the device 300 can include additional components not shown in FIG. 4A including an artificial intelligence accelerator.

In various alternatives, the processor 302 includes a central processing unit (CPU), a graphics processing unit (GPU), a CPU and GPU located on the same die, or one or more processor cores, wherein each processor core can be a CPU or a GPU. In various alternatives, the memory 304 is located on the same die as the processor 302, or is located separately from the processor 302. The memory 304 includes a volatile or non-volatile memory, for example, random access memory (RAM), dynamic RAM, or a cache.

The storage device 306 includes a fixed or removable storage means, for example, a hard disk drive, a solid state drive, an optical disk, or a flash drive. The input devices 308 include, without limitation, a keyboard, a keypad, a touch screen, a touch pad, a detector, a microphone, an accelerometer, a gyroscope, a biometric scanner, or a network connection (e.g., a wireless local area network card for transmission and/or reception of wireless IEEE 802 signals). The output devices 310 include, without limitation, a display, a speaker, a printer, a haptic feedback device, one or more lights, an antenna, or a network connection (e.g., a wireless local area network card for transmission and/or reception of wireless IEEE 802 signals).

The input driver 312 communicates with the processor 302 and the input devices 308, and permits the processor 302 to receive input from the input devices 308. The output driver 314 communicates with the processor 302 and the output devices 310, and permits the processor 302 to send output to the output devices 310. It is noted that the input driver 312 and the output driver 314 are optional components, and that the device 300 will operate in the same manner if the input driver 312 and the output driver 314 are not present. The output driver 316 includes an accelerated processing device (“APD”) 316 which is coupled to a display device 318. The APD accepts compute commands and graphics rendering commands from processor 302, processes those compute and graphics rendering commands, and provides pixel output to display device 318 for display. As described in further detail below, the APD 316 includes one or more parallel processing units to perform computations in accordance with a single-instruction-multiple-data (“SIMD”) paradigm. Thus, although various functionality is described herein as being performed by or in conjunction with the APD 316, in various alternatives, the functionality described as being performed by the APD 316 is additionally or alternatively performed by other computing devices having similar capabilities that are not driven by a host processor (e.g., processor 302) and provides graphical output to a display device 318. For example, it is contemplated that any processing system that performs processing tasks in accordance with a SIMD paradigm may perform the functionality described herein. Alternatively, it is contemplated that computing systems that do not perform processing tasks in accordance with a SIMD paradigm performs the functionality described herein.

FIG. 4B illustrates a graphical depiction of an artificial intelligence system 200 incorporating the example device of FIG. 4A. System 400 includes data 410, a machine 420, a model 430, a plurality of outcomes 440 and underlying hardware 450. System 400 operates by using the data 410 to train the machine 420 while building a model 430 to enable a plurality of outcomes 440 to be predicted. The system 400 may operate with respect to hardware 450. In such a configuration, the data 410 may be related to hardware 450 and may originate with the patient biometric monitoring and processing apparatus 102, for example. For example, the data 410 may be on-going data, or output data associated with hardware 450. The machine 420 may operate as the controller or data collection associated with the hardware 450, or be associated therewith. The model 430 may be configured to model the operation of hardware 450 and model the data 410 collected from hardware 450 in order to predict the outcome achieved by hardware 450. Using the outcome 440 that is predicted, hardware 450 may be configured to provide a certain desired outcome 440 from hardware 450.

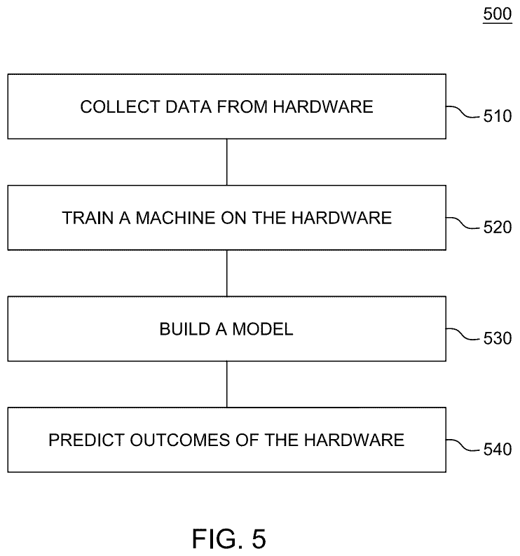

FIG. 5 illustrates a method 500 performed in the artificial intelligence system of FIG. 4B. Method 500 includes collecting data from the hardware at step 510. This data may include currently collected, historical or other data from the hardware. For example, this data may include measurements during a surgical procedure and may be associated with the outcome of the procedure. For example, the temperature of a heart (e.g., the heart 12) may be collected and correlated with the outcome of a heart procedure.

At step 520, method 500 includes training a machine on the hardware. The training may include an analysis and correlation of the data collected in step 510. For example in the case of the heart, the data of temperature and outcome may be trained to determine if a correlation or link exists between the temperature of the heart during the procedure and the outcome.

At step 530, method 500 includes building a model on the data associated with the hardware. Building a model may include physical hardware or software modeling, algorithmic modeling and the like, as will be described below. This modeling may seek to represent the data that has been collected and trained.

At step 540, method 500 includes predicting the outcomes of the model associated with the hardware. This prediction of the outcome may be based on the trained model. For example, in the case of the heart, if the temperature during the procedure between 97.7-100.2 produces a positive result from the procedure, the outcome can be predicted in a given procedure based on the temperature of the heart during the procedure. While this model is rudimentary, it is provided for exemplary purposes and to increase understanding of the present invention.

The mapping engine 101 herein provides the solutions by including a dense encoder-decoder network with a regularization term to reconstruct the shape of the left atrium from partial data, which is derived from catheter maneuvers. The mapping engine 101 can operate to train, build a model, and predict outcomes using certain algorithms. The algorithms of mapping engine 101 may be used to solve the trained model and predict outcomes associated with the hardware. By way of example, the algorithms of mapping engine 101 may be divided generally into classification, regression and clustering algorithms.

For example, a classification algorithm is used to identify the class of a dependent variable. In other words, given a particular value for a dependent variable that can be in one class of a set of classes, the classification algorithm determines which class the dependent variable belongs to. Thus, a classification algorithm is used to predict an outcome, from a set number of fixed, predefined outcomes. A classification algorithm may include naive Bayes algorithms, decision trees, random forest classifiers, logistic regressions, support vector machines, and k nearest neighbors.

Generally, a naive Bayes algorithm follows the Bayes theorem, and follows a probabilistic approach. As would be understood, other probabilistic-based algorithms may also be used, and generally operate using similar probabilistic principles to those described below for the exemplary naive Bayes algorithm.

Generally, a decision tree is a flowchart-like tree structure where each external node denotes a test on an attribute and each branch represents the outcome of that test. The leaf nodes contain the actual predicted labels (“classes”). The decision tree begins from the root of the tree with attribute values being compared until a leaf node is reached. A decision tree can be used as a classifier when handling high dimensional data and when little time has been spent behind data preparation. Decision trees may take the form of a simple decision tree, a linear decision tree, an algebraic decision tree, a deterministic decision tree, a randomized decision tree, a nondeterministic decision tree, and a quantum decision tree.

A random forest classifier is a committee of decision trees, where each decision tree has been fed a subset of the attributes of data and predicts on the basis of that subset. The modes of the actual predicted values of the decision trees are taken into account to provide an ultimate random forest answer. The random forest classifier, generally, alleviates overfitting, which is present in a standalone decision tree, leading to a much more robust and accurate classifier.

Logistic Regression is another algorithm for binary classification tasks. Logistic regression is based on the logistic function, also called the sigmoid function. This S-shaped curve can take any real-valued number and map it between 0 and 1 asymptotically approaching those limits. The logistic model may be used to model the probability of a certain class or event existing such as pass/fail, win/lose, alive/dead or healthy/sick. This can be extended to model several classes of events such as determining whether an image contains a cat, dog, lion, etc. Each object being detected in the image would be assigned a probability between 0 and 1 with the sum of the probabilities adding to one.

In the logistic model, the log-odds (the logarithm of the odds) for the value labeled “1” is a linear combination of one or more independent variables (“predictors”); the independent variables can each be a binary variable (two classes, coded by an indicator variable) or a continuous variable (any real value). The corresponding probability of the value labeled “1” can vary between 0 (certainly the value “0”) and 1 (certainly the value “1”), hence the labeling; the function that converts log-odds to probability is the logistic function, hence the name. The unit of measurement for the log-odds scale is called a log it, from logistic unit, hence the alternative names. Analogous models with a different sigmoid function instead of the logistic function can also be used, such as the probit model; the defining characteristic of the logistic model is that increasing one of the independent variables multiplicatively scales the odds of the given outcome at a constant rate, with each independent variable having its own parameter; for a binary dependent variable this generalizes the odds ratio.

In a binary logistic regression model, the dependent variable has two levels (categorical). Outputs with more than two values are modeled by multinomial logistic regression and, if the multiple categories are ordered, by ordinal logistic regression (for example the proportional odds ordinal logistic model). The logistic regression model itself models probability of output in terms of input and does not perform statistical classification (it is not a classifier), though it can be used to make a classifier, for instance by choosing a cutoff value and classifying inputs with probability greater than the cutoff as one class, below the cutoff as the other; this is a common way to make a binary classifier.

A support vector machine (SVM) may be used to sort the data with the margins between two classes as far apart as possible. This is called maximum margin separation. The SVM may account for the support vectors while plotting the hyperplane, unlike linear regression which uses the entire dataset for that purpose.

In regression algorithms, the output is a continuous quantity so regression algorithms may be used in cases where the target variable is a continuous variable. Linear regression is a general example of regression algorithms. Linear regression may be used to gauge genuine qualities (cost of houses, number of calls, all out deals and so forth) in view of the consistent variable(s). A connection between the variables and the outcome is created by fitting the best line (hence linear regression). This best fit line is known as regression line and spoken to by a direct condition Y=a*X+b. Linear regression is best used in approaches involving a low number of dimensions

Clustering algorithms may also be used to model and train on a data set. In clustering, the input is assigned into two or more clusters based on feature similarity. Clustering algorithms generally learn the patterns and useful insights from data without any guidance. For example, clustering viewers into similar groups based on their interests, age, geography, etc. may be performed using unsupervised learning algorithms like K-means clustering.

K-means clustering generally is regarded as a simple unsupervised learning approach. In K-means clustering similar data points may be gathered together and bound in the form of a cluster. One method for binding the data points together is by calculating the centroid of the group of data points. In determining effective clusters, in K-means clustering the distance between each point from the centroid of the cluster is evaluated. Depending on the distance between the data point and the centroid, the data is assigned to the closest cluster. The goal of clustering is to determine the intrinsic grouping in a set of unlabeled data. The ‘K’ in K-means stands for the number of clusters formed. The number of clusters, (basically the number of classes in which new instances of data may be classified), may be determined by the user. This determination may be performed using feedback and viewing the size of the clusters during training, for example.

K-means is used majorly in cases where the data set has points which are distinct and well separated, otherwise, if the clusters are not separated the modeling may render the clusters inaccurate. Also, K-means may be avoided in cases where the data set contains a high number of outliers or the data set is non-linear.

According to one or more embodiments, the mapping engine 101 can utilize ensemble learning algorithms. The ensemble learning algorithms of mapping engine 101 use multiple learning algorithms to obtain better predictive performance than could be obtained from any of the constituent learning algorithms alone. Ensemble learning algorithms perform the task of searching through a hypothesis space to find a suitable hypothesis that will make good predictions with a particular problem. Even if the hypothesis space contains hypotheses that are very well-suited for a particular problem, it may be very difficult to find a good hypothesis. Ensemble algorithms combine multiple hypotheses to form a better hypothesis. The term ensemble is usually reserved for methods that generate multiple hypotheses using the same base learner. The broader term of multiple classifier systems also covers hybridization of hypotheses that are not induced by the same base learner.

Evaluating the prediction of an ensemble typically requires more computation than evaluating the prediction of a single model, so ensembles may be thought of as a way to compensate for poor learning algorithms by performing a lot of extra computation. Fast algorithms such as decision trees are commonly used in ensemble methods, for example, random forests, although slower algorithms can benefit from ensemble techniques as well.

An ensemble is itself a supervised learning algorithm, because the ensemble can be trained and then used to make predictions. The trained ensemble, therefore, represents a single hypothesis. This hypothesis, however, is not necessarily contained within the hypothesis space of the models from which it is built. Thus, ensembles can be shown to have more flexibility in the functions they can represent. This flexibility can, in theory, enable them to over-fit the training data more than a single model would, but in practice, some ensemble techniques (especially bagging) tend to reduce problems related to over-fitting of the training data.

Empirically, ensemble algorithms tend to yield better results when there is a significant diversity among the models. Many ensemble methods, therefore, seek to promote diversity among the models they combine. Although non-intuitive, more random algorithms (like random decision trees) can be used to produce a stronger ensemble than very deliberate algorithms (like entropy-reducing decision trees). Using a variety of strong learning algorithms, however, has been shown to be more effective than using techniques that attempt to dumb-down the models in order to promote diversity.

The number of component classifiers of an ensemble has a great impact on the accuracy of prediction. A priori determining of ensemble size and the volume and velocity of big data streams make this even more crucial for online ensemble classifiers. A theoretical framework suggests that there are an ideal number of component classifiers for an ensemble such that having more or less than this number of classifiers would deteriorate the accuracy. The theoretical framework shows that using the same number of independent component classifiers as class labels gives the highest accuracy.

Some types of ensembles include Bayes optimal classifier, bootstrap aggregating (bagging), boosting, Bayesian model averaging, Bayesian model combination, bucket of models and stacking.

According to one or more embodiments, the mapping engine 101 can include one or more neural networks. A neural network is a network or circuit of neurons, or in a modern sense, an artificial neural network, composed of artificial neurons or nodes. The connections of the biological neuron are modeled as weights. A positive weight reflects an excitatory connection, while negative values mean inhibitory connections. Inputs are modified by a weight and summed using a linear combination. An activation function may control the amplitude of the output. For example, an acceptable range of output is usually between 0 and 1, or it could be −1 and 1.

These artificial networks may be used for predictive modeling, adaptive control and applications and can be trained via a dataset. Self-learning resulting from experience can occur within networks, which can derive conclusions from a complex and seemingly unrelated set of information.

For completeness, a biological neural network is composed of a group or groups of chemically connected or functionally associated neurons. A single neuron may be connected to many other neurons and the total number of neurons and connections in a network may be extensive. Connections, called synapses, are usually formed from axons to dendrites, though dendrodendritic synapses and other connections are possible. Apart from the electrical signaling, there are other forms of signaling that arise from neurotransmitter diffusion.

Artificial intelligence, cognitive modeling, and neural networks are information processing paradigms inspired by the way biological neural systems process data. Artificial intelligence and cognitive modeling try to simulate some properties of biological neural networks. In the artificial intelligence field, artificial neural networks have been applied successfully to speech recognition, image analysis and adaptive control, in order to construct software agents (in computer and video games) or autonomous robots.

In general, a neural network is a network or circuit of neurons, or in a modern sense, an artificial neural network (ANN), composed of artificial neurons or nodes or cells.

For example, an ANN involves a network of processing elements (artificial neurons) which can exhibit complex global behavior, determined by the connections between the processing elements and element parameters. One classical type of artificial neural network is the recurrent Hopfield network. These connections of the network or circuit of neurons are modeled as weights. A positive weight reflects an excitatory connection, while negative values mean inhibitory connections. Inputs are modified by a weight and summed using a linear combination. An activation function may control the amplitude of the output. For example, an acceptable range of output is usually between 0 and 1, or it could be −1 and 1. In most cases, the ANN is an adaptive system that changes its structure based on external or internal information that flows through the network.

In more practical terms, neural networks are non-linear statistical data modeling or decision-making tools that can be used to model complex relationships between inputs and outputs or to find patterns in data. Thus, ANNs may be used for predictive modeling and adaptive control applications, while being trained via a dataset. Note that self-learning resulting from experience can occur within ANNs, which can derive conclusions from a complex and seemingly unrelated set of information. The utility of artificial neural network models lies in the fact that they can be used to infer a function from observations and also to use it. Unsupervised neural networks can also be used to learn representations of the input that capture the salient characteristics of the input distribution, and more recently, deep learning algorithms, which can implicitly learn the distribution function of the observed data. Learning in neural networks is particularly useful in applications where the complexity of the data (e.g., the biometric data) or task (e.g., monitoring, diagnosing, and treating any number of various diseases) makes the design of such functions by hand impractical.

Neural networks can be used in different fields. Thus, for the artificial intelligence system 400, the machine learning and/or the artificial intelligence algorithms therein can include neural networks that are divided generally according to tasks to which they are applied. These divisions tend to fall within the following categories: regression analysis (e.g., function approximation) including time series prediction and modeling; classification including pattern and sequence recognition; novelty detection and sequential decision making; data processing including filtering; clustering; blind signal separation, and compression. For example, application areas of ANNs include nonlinear system identification and control (vehicle control, process control), game-playing and decision making (backgammon, chess, racing), pattern recognition (radar systems, face identification, object recognition), sequence recognition (gesture, speech, handwritten text recognition), medical diagnosis and treatment, financial applications, data mining (or knowledge discovery in databases, “KDD”), visualization and e-mail spam filtering. For example, it is possible to create a semantic profile of patient biometric data emerging from medical procedures.

According to one or more embodiments, the neural network can implement a long short-term memory neural network architecture, a convolutional neural network (CNN) architecture, or other the like. The neural network can be configurable with respect to a number of layers, a number of connections (e.g., encoder/decoder connections), a regularization technique (e.g., dropout); and an optimization feature.

The long short-term memory neural network architecture includes feedback connections and can process single data points (e.g., such as images), along with entire sequences of data (e.g., such as speech or video). A unit of the long short-term memory neural network architecture can be composed of a cell, an input gate, an output gate, and a forget gate, where the cell remembers values over arbitrary time intervals and the gates regulate a flow of information into and out of the cell.

The CNN architecture is a shared-weight architecture with translation invariance characteristics where each neuron in one layer is connected to all neurons in the next layer. The regularization technique of the CNN architecture can take advantage of the hierarchical pattern in data and assemble more complex patterns using smaller and simpler patterns. If the neural network implements the CNN architecture, other configurable aspects of the architecture can include a number of filters at each stage, kernel size, a number of kernels per layer.

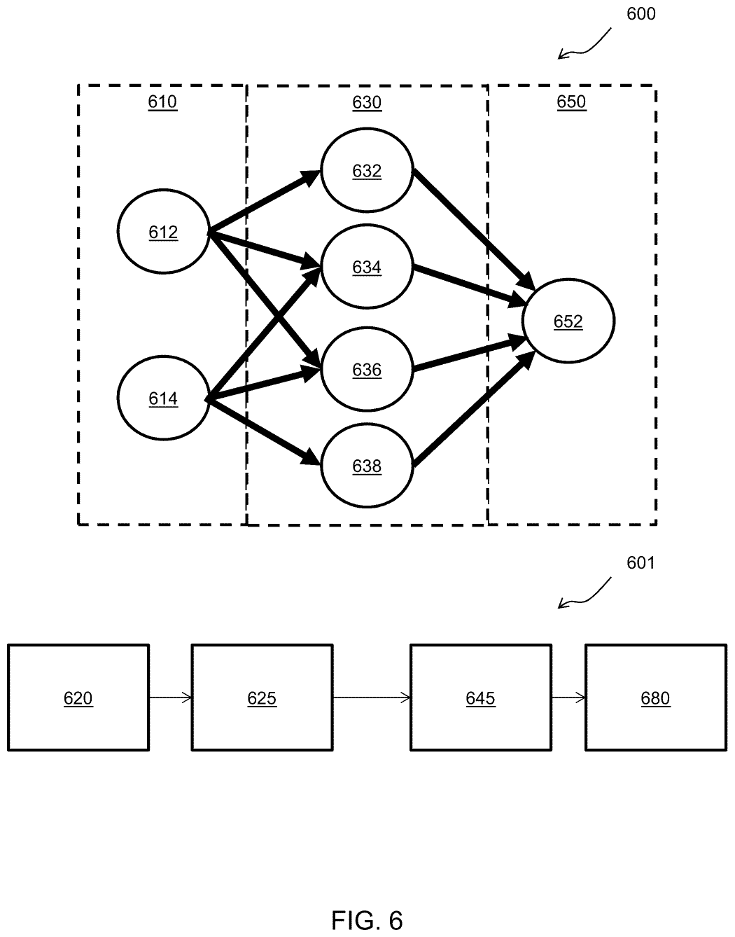

Turning now to FIG. 6, an example of a neural network 600 and a block diagram of a method 601 performed in the neural network 600 are shown according to one or more embodiments. The neural network 600 of FIG. 6 may be implemented in hardware. The neural network 600 operates to support implementation of the machine learning and/or the artificial intelligence algorithms (e.g., as implemented by the mapping engine 101) described herein. The neural network 600 can be implemented in hardware, such as the machine 420 and/or the hardware 450 of FIG. 4B. As indicated herein, the description of FIG. 6 is made with reference to other figures for ease of understanding where appropriate.

In an example operation, the mapping engine 101 collects the data 410 from the hardware 450. In the neural network 600, an input layer 610 is represented by a plurality of inputs (e.g., inputs 612 and 614 of FIG. 6). With respect to block 620 of the method 601, the input layer 610 receives the inputs 612 and 614. The inputs 612 and 614 can include biometric data. For example, the collecting of the data 410 can be an aggregation of biometric data (e.g., BS ECG data, IC ECG data, and ablation data, along with catheter electrode position data), from one or more procedure recordings of the hardware 450 into a dataset (as represented by the data 410).

At block 625 of the method 601, the neural network 600 encodes the inputs 612 and 614 utilizing any portion of the data 410 (e.g., the dataset and predictions produced by the artificial intelligence system 400) to produce a latent representation or data coding. The latent representation includes one or more intermediary data representations derived from the plurality of inputs. According to one or more embodiments, the latent representation is generated by an element-wise activation function (e.g., a sigmoid function or a rectified linear unit) of the mapping engine 101 of FIG. 1. As shown in FIG. 6, the inputs 612 and 614 are provided to a hidden layer 630 depicted as including nodes 632, 634, 636, and 638. The neural network 600 performs the processing via the hidden layer 630 of the nodes 632, 634, 636, and 638 to exhibit complex global behavior, determined by the connections between the processing elements and element parameters. Thus, the transition between layers 610 and 630 can be considered an encoder stage that takes the inputs 612 and 614 and transfers it to a deep neural network (within layer 630) to learn some smaller representation of the input (e.g., a resulting the latent representation).

The deep neural network can be a CNN, a long short-term memory neural network, a fully connected neural network, or combination thereof. The inputs 612 and 614 can be intracardiac ECG, body surface ECG, or intracardiac ECG and body surface ECG. This encoding provides a dimensionality reduction of the inputs 612 and 614. Dimensionality reduction is a process of reducing the number of random variables (of the inputs 612 and 614) under consideration by obtaining a set of principal variables. For instance, dimensionality reduction can be a feature extraction that transforms data (e.g., the inputs 612 and 614) from a high-dimensional space (e.g., more than 10 dimensions) to a lower-dimensional space (e.g., 2-3 dimensions). The technical effects and benefits of dimensionality reduction include reducing time and storage space requirements for the data 410, improving visualization of the data 410, and improving parameter interpretation for machine learning. This data transformation can be linear or nonlinear. The operations of receiving (block 620) and encoding (block 625) can be considered a data preparation portion of the multi-step data manipulation by the mapping engine 101.

At block 645 of the method 610, the neural network 600 decodes the latent representation. The decoding stage takes the encoder output (e.g., the resulting the latent representation) and attempts to reconstruct some form of the inputs 612 and 614 using another deep neural network. In this regard, the nodes 632, 634, 636, and 638 are combined to produce in the output layer 650 an output 652, as shown in block 660 of the method 601. That is, the output layer 690 reconstructs the inputs 612 and 614 on a reduced dimension but without the signal interferences, signal artifacts, and signal noise. Examples of the output 652 include cleaned biometric data (e.g., clean/denoised version of IC ECG data or the like). The technical effects and benefits of the cleaned biometric data include enabling more accurate monitor, diagnosis, and treatment any number of various diseases.

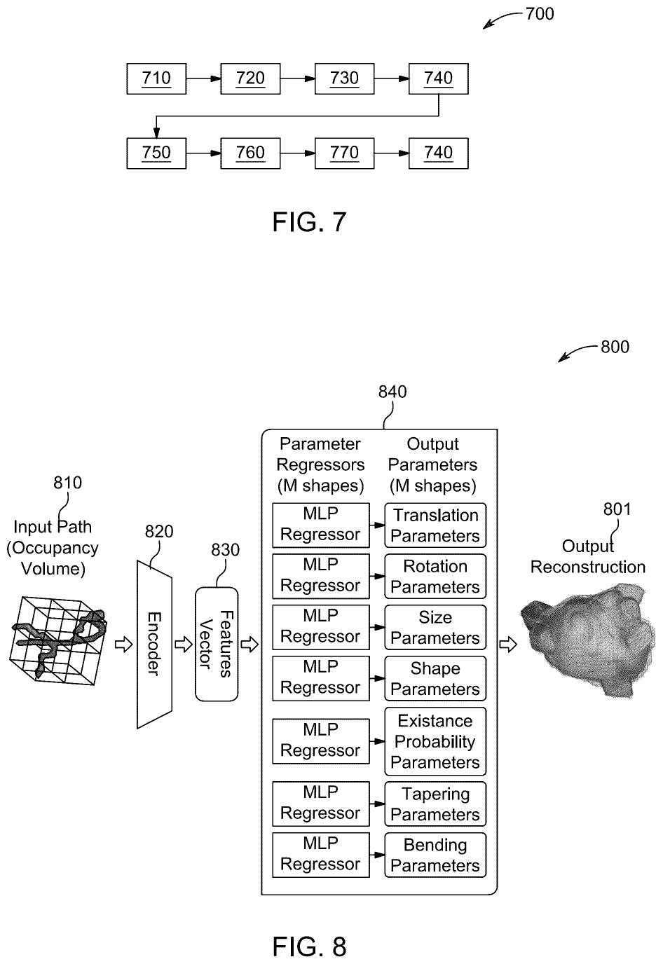

Turning now to FIG. 7, a method 700 (e.g., performed by the mapping engine 101) is illustrated according to one or more exemplary embodiments. Generally, the method 700 extracts corresponding catheter trajectories, per atrium case, to approximate realistic reconstruction of an anatomical structure. By way of example, the method 700 provides superquadratics neural network reconstruction of the anatomical structure (e.g., a LA) using sparse catheter paths. That is, the method 700 addresses a need for early visualization that guide catheters by generating maps, which include the superquadratics neural network reconstruction of an endocardial surface of the LA using one or more portions of a catheter traversal path (e.g., initial bearings path).

The method 700 begins at block 710, where the mapping engine 101 performs a pre-training phase. The pre-training phase includes training one or more neural networks to provide one or more pre-trained neural networks. To train the one or more neural networks, the mapping engine 101 obtains shapes, trajectories, or both, where the shapes and trajectories are defined by output primitives. For example, some of the inputs for training can include one or more geometric shape families, as well as any identified corresponding shapes and specific primitives within those geometric shape families. That is, to model anatomical shape composition, the mapping engine 101 can use primitives, such as translation, rotation, size, shape, existence probability, tapering, and bending, as parameters of the neural network. In addition, the inputs can include trajectories (e.g., of a catheter). In summary, the inputs to the pre-training phase include catheter trajectories and anatomical shapes that represent the portion of a left atrial anatomical structure observed while generating the catheter trajectories. The pre-training phase generates the pre-trained neural networks that are able to generate early left atrium visualizations based on catheter paths.

At block 720, the mapping engine 101 acquires a dataset. The dataset can include one or more geometric shape families, such as 3D atria shapes constructed from superquadratics.

At block 730, the mapping engine 101 acquires catheter trajectories. The trajectories can be received in real-time during an ablation procedure. According to one or more embodiments, the trajectories can be an input path representing an occupancy volume of a portion of a catheter traversing in the vicinity of a left atrium. Upon receipt of the input path, the mapping engine 101 encodes the input path to produce a feature vector used to identify attributes, transformations, and existence probability of primitives of a left atrium shape, as further described herein. The mapping engine 101 corresponds the catheter trajectories to the dataset, such as 3D atria shapes, and choses a corresponding pre-trained network.

Note that physicians follow an initial bearings path that traverses known anatomical landmarks, such as the four pulmonary vein ostia within three minutes. The technical effects and benefits of the mapping engine 101 include enabling a guide for catheters with difficulty sampling surfaces of anatomy, such as single-shot catheters.

At block 740, the mapping engine 101 trains the chosen pre-trained network. That is, the pre-trained network is further trained based on the acquired dataset and the catheter trajectories. The further trained pre-trained network can be considered a trained neural network. According to one or more embodiments, the pre-trained network is trained on the input path using 3D atria shapes constructed from superquadratics. In other words, the shapes that comprise the entire 3D model generated by the pre-trained network are “superquadratic shapes.” A superquadratic shape is defined by Equation 1, which is set forth below. In an example, for training, the mapping engine 101 utilizes a set of acquired left atrial CT shapes, to train for LA anatomy variation, with four pulmonary veins representing most of a population dataset. According to one or more embodiments, the mapping engine 101 utilizes a software simulator to create and augment a database storing the dataset and the catheter trajectories, thus creating a “constructed” set of catheter paths and left atrial shapes to supplement the real, acquired data.

At block 750, which is a part of the reconstruction operation, the mapping engine 101 approximates the atrium shape. According to one or more embodiments, once the pre-trained network is trained, the mapping engine 101 utilizes that suggested network to accurately approximate the atrium shape based on a given input trajectory (e.g., a measured catheter trajectory).

At block 760, the mapping engine 101 generates an output. For instance, the output can be the output from trained neural network, generated at block 750. The output can be a 3D model that comprises an early visualization of the anatomy being scanned.

At block 770, the mapping engine 101 displays the output (e.g., the early visualization). According to one or more embodiments, by displaying the output, the mapping engine 101 can provide the 3D model as a representation of the anatomical structure in a meaningful way to the physician and/or the technician. By way of example, the representation of the anatomical structure in the meaningful way by the mapping engine 101 can include enabling the physician and/or the technician through the 3D model to perform the procedure or operate based on inferences that can trigger or enable further mapping (e.g., via other conventional technologies).

In some implementations, at block 780, the mapping engine 101 compares the 3D model. The mapping engine 101 can compare the 3D model across several network solutions for the 3D atrium reconstruction. The mapping engine 101, in this regard, demonstrates that the generated 3D model shortens procedure time, and does so with real-time performance. The reconstructed openings and orientations of the pulmonary veins in the generated 3D model have minimal errors and the anatomical parts are easily identifiable. The technical effects and benefits of the mapping engine 101 include reducing the mapping time while maintaining anatomical accuracy by mapping the endocardial surface of the left atrium (LA) using a portion of the catheter traversal path (initial bearings path) to provide early visualization, especially for the pulmonary veins and important anatomy parts.

According to one or more embodiments, the mapping engine 101 generates additional training data to overcome the lack of data, the mapping engine 101 utilizing a LA instance generator to create left atrial anatomical shapes and an algorithm to create clinically feasible simulated sparse catheter paths, for each generated LA shape.

In summary, the technique of FIG. 7 involves training one or more neural networks to output a reconstructed LA shape based on an input catheter path. The output LA shape includes or is derived from superquadratics shapes (defined by Equation 1). In the example of the present disclosure, the input representation is a catheter path that describes a measured path of a catheter through anatomy. The primitive representation is a parameterized description of the left atrium anatomy. The parameters represent geometric aspects of a set of primitives (e.g., shapes) of the left atrium anatomy. The training regime of the mapping engine 101 allows the mapping engine 101 to incorporate LA anatomy knowledge or “priors”. Additional details about how training incorporates such “priors” or knowledge about LA anatomy are provided below. The resulting representation generated by the mapping engine 101 provides a flexible framework that enables semantic segmentation of the LA into different parts, allows for post-inference editing, and ensures the anatomical correctness of the reconstruction. Thus, the mapping engine 101 allows physicians to incorporate clinically relevant information, such as the PVLS and LAA separation, into the reconstruction process. Put differently, the semantic representation of the left atrial anatomy—the representation as a parameterized three-dimensional model—allows for editing of this generated representation by allowing editing of the parameterized three-dimensional model. Data from clinical trials involving human subjects have shown that the reconstruction operations by the mapping engine 101 produce more accurate results than those obtained using previous DED and V-Net solutions. In some examples, a technique utilizing steps 730, 740, and 750 (and not the other described steps) are possible. In various examples, techniques including any subset of the steps of the method 700 are contemplated. In particular, techniques that include training the neural network based on input data (e.g., obtained catheter trajectories and actual LA shapes) and then performing reconstruction are contemplated. In addition, in some examples, techniques that include only training steps (e.g., 710-740 or 720-740) or only reconstruction steps (e.g., 750 or 750-760) are contemplated.

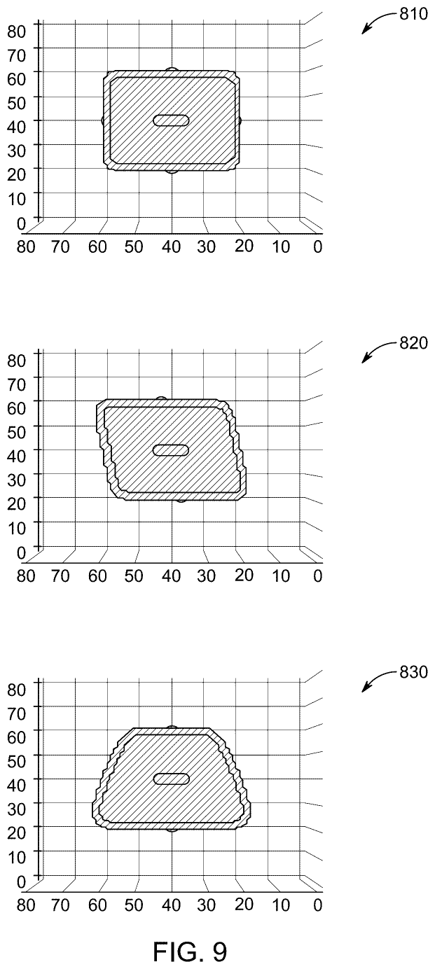

FIG. 8 depicts a system 800 according to one or more embodiments. In some examples, the operations described as being performed by the system 800 of FIG. 8 represent at least operation 750 of FIG. 7. The system 800 is an example schematic of at least a portion of the mapping engine 101. Operation of the mapping engine 101 provides a reconstruction 901 of the left atrial shape using a superquadrics network (SQNet), which leverages medical knowledge about a general shape of the left atrium (LA). That is, the mapping engine 101 learns to represent a shape of the LA by transforming a set of geometric primitives that are generalized ellipsoids (superquadratics). The union of these primitive shapes defines the interior of the LA, while the boundary is the surface.

An input path 810 is provided to the system 800. Herein, the term “input path” is used interchangeably with the term “catheter traversal path.” The input traversal path is a recorded path of a catheter or other instruction. In various examples, this path is obtained in any technically feasible manner, such as via electro-anatomical mapping, described elsewhere herein. The input path 810 is encoded 820 using a convolutional neural network (“CNN”), which produces a feature vector 830 used by the geometry regression network 840 to independently regress the attributes, transformations, and existence probability of the primitives that characterize the left atrial shape. The mapping engine 101 can include and utilize a modified version of the SQNet called the Left Atrial Shape Reconstruction Using Super Quadrics Network (LASQNet), specifically designed for left atrial shape reconstruction. Accordingly, an overall structure of the LASQNet can be similar to the SQNet. More specifically, the geometry regression network 840 is a network that produces an output 3D model that includes a plurality of superquadratic shapes, where the network is trained to incorporate and thus reproduce LA anatomy.

Regarding how the primitives that characterize the left atrial shape are defined (e.g., the format of the shapes output by the geometry regression network 840), the shapes of the primitives are fully defined using analytical formulas that allow the mapping engine 101 to determine, for any given 3D point in space, whether it is inside the shape, on the surface, or outside the shape. These formulas also enable approximation of the distance of the point from a surface of the shape. A point \vec{x}∈R³ (e.g., a three-dimensional coordinate) is considered to be inside (or on the boundary of) a primitive if the following inequality (strongly) holds according to Equation 1, where a and ϵ control the shape size and shape, respectively. The parameters α_{X} and ϵ_{Y} (where X is 1, 2, and 3 and Y is 1 or 2, as shown below) describe superquadratic geometry (that is, the geometry of the shapes being modeled).

$$F ( x , y , z ) = {( {( \frac{x}{{∝}_{1}} )}^{\frac{2}{{∈}_{2}}} + {( \frac{y}{{∝}_{2}} )}^{\frac{2}{{∈}_{2}}} )}^{\frac{{∈}_{2}}{{∈}_{1}}} + {( \frac{z}{{∝}_{3}} )}^{\frac{2}{{∈}_{1}}} \leq 1 \qquad Equation 1$$

The distance from a 3D point to the surface of a shape can be approximated using Equation 2, which gives the distance between the intersection of the radius vector of \vec{x} and the surface, and the point \vec{x} itself.

$$d ( \vec{x} ) = \vert \vec{x} \vert \vert 1 - {F ( \vec{x} )}^{\frac{{∈}_{1}}{2}} \vert \qquad Equation 2$$

Equation 2 provides an upper bound for the Euclidean distance and is equal to zero if the point is on the surface. Using this Equation 2, the mapping engine 101 can define a function ASD(\vec{x}) for each shape that bounds the signed distance transform (in absolute value) for every point. Specifically, see Equation 3.

$$A S D ( \vec{x} ) = Sign ( F ( \vec{x} ) - 1 ) \cdot d ( \vec{x} ) \qquad Equation 3$$

A function (See Equation 4) indicates whether a point is inside, on the boundary, or outside all of the shape primitives, and provides the approximate distance to the surface of the shapes.

$$Q ( \vec{x} ) = {min}_{i} A S D ( \vec{x} , i ) \qquad Equation 4$$

According to one or more embodiments, training operations train the neural network of the mapping engine 101 to generate a 3D model of a left atrium using a number of techniques. These techniques including incorporating prior knowledge of specific shapes appropriate for the anatomy of a left atrium. The techniques also include training the neural network to apply a set of transformations to make the resulting shape more realistic (e.g., loss functions are used to control different scenarios in the optimization process). The neural network is also trained to apply an overlap constraint to keep a resulting shape connected. The neural network is trained using a particular loss function that is combined from different loss parts, and training the neural network is done using certain stages of the optimization and post-inference optimization process (e.g., which can fix constraints).

Some of the above operations also define what occurs in reconstruction. For example, during reconstruction, the mapping engine 101 accepts an input catheter path and obtains a set of output parameters that define the geometry of the reconstructed left atrium. These output parameters include the translation parameters, rotation parameters, size parameters, shape parameters, existence probability parameters, tapering parameters, and bending parameters. For each of a set of shapes that are deemed to be included in the reconstruction of the atrium based on a priori knowledge, the existence probabilities define whether such shapes are included in the reconstruction. These “a priori shapes” are described below as “chosen shapes.” The translation parameters, rotation parameters, size parameters, and shape parameters define the shape and size of the chosen shapes as well as the position and rotation of such shapes. The tapering parameters and bending parameters define tapering and bending applied to each of the shapes. As can be seen, the reconstruction generates parameters that define 3D geometry of a reconstructed left atrium based on an input catheter path.

With respect to controlling chosen shapes, the mapping engine 101 uses the SQNet to incorporate anatomical knowledge into the optimization (a part of training) process by decomposing the atria shape into primitives. The knowledge of the anatomical parts that make up an atrium can guide the optimization process of the mapping engine 101 to learn shapes similar to human understanding of anatomy. The LA includes an ellipsoid-like main body with four pulmonary veins attached at the roof, a bulge (the appendage) below the left pulmonary veins, and a narrowing (the valve) at the bottom. To guide the optimizer to use these building blocks, for training (e.g., step 740), the mapping engine 101 manipulates the inferred primitive sizes of the neural network. More specifically, the SQNet predicts the size of each axis for each primitive. The mapping engine 101 applies a linear transformation aσ(x)+b (where σ is a non-linearity function), with different parameters for each primitive, to control the range of the primitive size. The scaling directly affects the gradient and thus the manner in which training occurs, thus controlling the optimization. In other words, the parameters set for this linear transformation control optimization of the neural network for training, causing the neural network to learn the appropriate parameters for the shapes that make up the left atrium. For the LA body, the mapping engine 101 sets the value of a for the first two shapes of the generated 3D model of the left atrium to a large value and for two other shapes of the generated 3D model to a medium value (i.e., smaller than the large value). For the remaining shapes of the left atrium, intended to describe the pulmonary veins, the appendage, and other small structures, the mapping engine 101 sets the range of a to be a smaller value than the medium value (e.g., between 1 and 3 voxels, where a voxel is the smallest unit of volume describable in the 3D model). To encourage the solution to include the LA body and prevent convergence to the trivial no-shape solution, the mapping engine 101 sets the existence probability of the first shape to 1. It should be understood that applying these linear transformations and setting these parameters occurs during training to cause the neural network of the mapping engine 101 to be trained to generate the desired shapes.

With respect to transformations, each primitive is defined in a local coordinate system where its center is at the origin. Points in the primitive coordinate system undergo a series of invertible non-linear transformations, followed by a rigid rotation Ri and translation Ti to a point in the “world” coordinates. The rigid transformation is the rotation Ri and translation Ti and is represented using a quaternion and a translation vector. To compute the value of Q(\vec{x}) for a point (\vec{x}) in the world coordinates, each primitive undergoes the same sequence of transformations, but in reverse order and inverted.

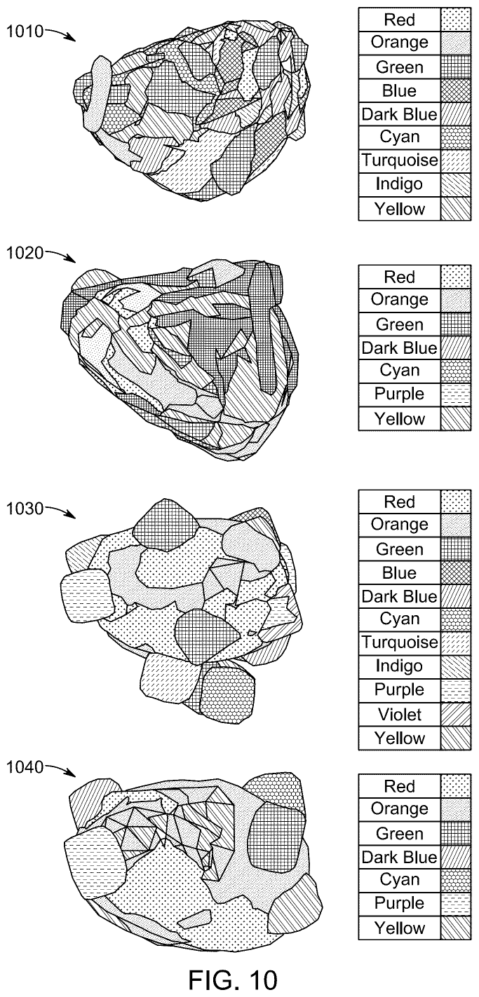

FIG. 9 depicts transformations 910, 920, and 930 according to one or more embodiments. In some cases, the mapping engine 101 uses the tapering and bending transforms (e.g., as described above). The tapering transform transforms the boundary of each coordinate axis of a particular shape as a polynomial function of the reference axis z. For each coordinate axis a∈x, y, with a shape size of the z axis S_{z}, the second degree tapering is defined in Equation 5, while it is the identity function for the z coordinate. k, w are learned tapering parameters (i.e., parameters output by the neural network of the mapping engine 101).

$${T}_{a ∈ \{ x , y \}} = a ( 1 + {k}_{a} \frac{z}{{S}_{z}} - {w}_{a} \frac{z}{{S}_{z}} ) \qquad Equation 5$$

The bending transform translates each of the two endpoints of the shape by a 2D translation vector. The rest of the space is bent according to these points, linearly by the z values. As shown in Equation 6, β, γ are the learned bending parameters (i.e., parameters output by the neural network of the mapping engine 101). Note that the field function filters z values outside the shape.

$${β}_{a ∈ \{ x , y \}} = a + {β}_{a} \frac{z}{{S}_{z}} + {γ}_{b} \frac{1 - z}{{S}_{z}} \qquad Equation 6$$

Losses represent aspects of training. During training, the mapping engine 101 attempts to correct its own weights by comparing its actual output to the desired output. The comparison is performed using one or more loss functions. The loss functions define the manner in which and the degree to which the weights of the neural network deviate from the “desired” output. In various examples, the output of the neural network is the 3D model generated in response to the input catheter path and the “desired output” is the recorded actual geometry that is recorded for the input catheter path. With respect to losses, the mapping engine 101 uses one or more loss parts described herein. A portion of the overall loss function is referred to as a “loss part” herein. A combination (e.g., sum) of the loss parts defines the overall loss function. Several loss parts are now described.

The parameter pᵢ indicates the probability of primitive i being part of the output shape. The mean shape loss Lₘₛₑ is the sum of these probabilities (with some weight) to keep the solution sparse. Given a list of surface points, the point cloud to primitives loss L_{pcl→prim} is defined as the expected distance (over primitive existence probability) from each point to the nearest primitive. This cost ensures that the surface of the primitives is close to the target surface. The complementary form of this cost is the primitive to surface point cloud L_{prim→pcl}, which measures the distance from each point on the primitive surface to the closest point in the surface point cloud, and averages this over all points in the primitive. This is also taken by expectation over primitive existence probability. These last two costs are computed by sampling points from the surfaces of the primitives. To improve the accuracy of the optimization process in the fine details of the output shape, the mapping engine 101 uses three additional losses that are only applied to the active set of shapes (those with pᵢ>0.5). The three additional losses include an inside-outside loss, a surface boundary loss, and an overlap constraint loss.

According to one or more embodiments, an inside-outside loss aims to minimize the error by sampling points in the space and defining an in-loss and out-loss for each point that is misclassified as being inside or outside, respectively as shown in Equations 7 and 8, where CE is the cross entropy function.

$$InLoss ( \vec{x} ) = C E ( σ ( - Q ( \vec{x} ) ) , 1 ) \qquad Equation 7$$

$$OutLoss ( \vec{x} ) = C E ( σ ( - Q ( \vec{x} ) ) , 0 ) \qquad Equation 8$$

The in loss is computed for points with the label inside, while the out loss is computed for points with the outside label. The mapping engine 101 uses the same number of points for each group.

According to one or more embodiments, regarding a surface boundary loss, the function Q (X) is an upper bound for the signed distance inside the shape, since the distance from the surface of the union of the shape is greater than each primitive. This means that a point at the intersection of two primitives can be assigned a distance that is much lower than the actual distance. The mapping engine 101 defines an inside indicator function as IIF(\vec{x})=max(Sign(−Q(\vec{x})),0). This function is 1 if the point is inside (not on the surface) and 0 otherwise. This function has subgradients, but only gets a nonzero gradient value at the transition point. The sum exceeding count is defined as in Equation 9, which counts the number of surface points, denoted as ∂s, that are inside the shape.

$$S E C = {\sum }_{\vec{x} ∈ \partial s} ( I I F ( \vec{x} ) ) \qquad Equation 9$$

The mean exceeding field (MEF) is defined in Equation 10, where ϵ is a small constant. The MEF provides the gradient for each point to push it outside the interior of the shape. In practice, the mapping engine 101 minimizes both SEC and MEF.

$$M E F = {\sum }_{\vec{x} ∈ \partial s} \frac{max ( - Q ( \vec{x} ) , 0 )}{S E C + ∈} \qquad Equation 10$$

According to one or more embodiments, regarding an overlap constraint loss, the use of shape primitives with implicit field representations does not ensure that the generated shape will be connected. To check for connectivity, a connectivity graph must be constructed by comparing each primitive with all others. If the graph has only one connected component, the constraint is satisfied. A naive implementation of this process requires

$$O ( \frac{{n}^{2}}{2} )$$

comparisons, while more efficient algorithms such as disjoint-set trees can reduce complexity but are difficult to implement in a differentiable manner. In order to enforce overlap between the exterior primitives and the main body of the shape (assuming the main body primitives are known), the mapping engine 101 defines a constraint using a field defined in Equation 11, where B is the set of body primitives (e.g., primitives of the left atrium).

$$BodyQ ( \vec{x} ) = {min}_{i ∈ B} {F}_{i} \cdot Sign ( F ( \vec{x} ) - 1 ) \qquad Equation 11$$

The field is calculated for a set of sampled surface points from the rest of the primitives (translated to world coordinates). The function IBF(\vec{x})=max(Sign(−BodyQ(\vec{x})),0) returns 1 if the point is inside (or on the surface) of the body set and 0 otherwise. The mean field of the sampled primitive in the body field, MDᵢ(Sᵢ)=mean_{\vec{x}∈S}_{ᵢ}(BodyQ(\vec{x})), decreases as the sampled points move closer to the inside of the body. The overlap ratio for primitive is defined in Equation 12, which measures the fraction of the sampled points inside the body.

$$i , {RO}_{i} ( {S}_{i} ) = \frac{{\sum }_{\vec{x} ∈ {S}_{i}} I B F ( \vec{x} )}{\vert {S}_{i} \vert} \qquad Equation 12$$

The overlap loss is defined as OL=Σᵢmax(ρ−ROᵢ,0)·MDI, where ρ is the required overlap percentile. This cost requires each exterior primitive to have an overlap ratio of at least ρ, while the MDᵢ term provides the gradient direction when the constraint is active.

According to one or more embodiments, regarding a total cost function: The target cost function of the network is a weighted linear combination of the previously mentioned losses. The weights are hyper-parameters of the optimization stage. For example, see Equation 13.

$$L = {w}_{p c l \to prim} {L}_{pcl \to p r i m} + {w}_{prim \to pcl} {L}_{prim \to p c l} + {w}_{par} {L}_{par} + {w}_{mse} {L}_{mse} + {w}_{inl} INLoss + {w}_{outl} OUTLoss + {w}_{s ec} S E C + {w}_{mef} M E F + {w}_{ol} O L \qquad Equation 13$$

The cost is evaluated by sampling. Each primitive is sampled by N points (for example, by using a fast sampler). In addition, the mapping engine 101 samples KᵢₙK_{surface},Kₒᵤₜ points on the target interior, surface and exterior, to have the target points to the different losses.

According to one or more embodiments, regarding learning in stages, during the optimization process, the network parameters are updated based on the gradients in a somewhat greedy manner. This process typically converges to a valid solution within a few epochs. Once the solution approaches a valid one, some of the inferred parameters tend to remain stable. In the case of LA reconstruction, the choice of active shapes and the sizes of these shapes tend to remain constant after several epochs. These properties result in the ability to modify the optimization process by altering the weights of the loss terms, adding additional cost terms, and incorporating prior knowledge about the shapes. In a first stage, the mapping engine 101 runs the optimization process until convergence without using the bending transformation, as it introduces instability to the optimizer. As a final step, the mapping engine 101 introduces the bending transformation and the overlap constraint loss to the optimization process for fine-tuning.

According to one or more embodiments, regarding post inference optimization, in FAM (“fast anatomical mapping”—a technique for mapping anatomy) and other reconstructions, the PVLS and the appendage can become fused together. The ridge between these two parts is an important anatomical feature, and it is important to ensure that they are separated in the reconstruction. To address this issue, the mapping engine 101 provides an additional cost term that can be used to separate these two anatomical parts in a framework. Let ^{→}μls and ^{→}μapp be the translation vectors of the primitives describing the PVLS and the appendage, respectively. The function InAppQ(\vec{x})=(Fₐₚₚ−1)·Sign(Fₐₚₚ−1) measures the signed field within the appendage, with the distance proportional to the dimensions of the appendage axes. Given the distance vector \vec{D}li→app=\vec{82}app−\vec{μ}ls, the separation constraint with distance β is defined in Equation 14.

$$Sep ( β ) = \frac{min ( max ( max x ∈ \partial l s ( - InAppQ ( x ) + β ) , 0 ) , 1 )}{\vert \vec{D} li \to app \vert} \qquad Equation 14$$

This cost only assigns a positive value when the PVLS intersects the appendage, and the value is inversely proportional to the distance vector to directly relate the gradient of this cost to the center location of the primitives. This cost function can be referred to as the “Appendage Separation Cost”. Although the overlap constraint is introduced in a soft manner during the training process, it is possible for the constraint to be violated in the inferred result. To address this issue, the mapping engine 101 implements a method for performing a local optimization fix on the inferred LA sample. After inferring the LA sample, the mapping engine 101 manipulates the resulting shape parameter vector using a local optimization procedure, with the cost function being a linear combination of the Appendage Separation Cost and the overlap constraint loss. In some examples, the mapping engine 101 uses the LBFGS optimizer to modify the shape parameter vector (that is, the set of parameters, described above, that define the shapes of the reconstruction) according to the cost.

According to one or more embodiments, operational examples of the mapping engine are provided herein. For example, the results of experiments on reconstructing the shape of the left atrium using LASQNet are provided. As discussed herein, the mapping engine 101 demonstrates an effectiveness of the LASQNet shape guidance in obtaining a valid solution. Further, the mapping engine 101 experiments on synthetic path reconstruction using human clinical cases, provide example use cases for the anatomical representation of the LASQNet (including semantic segmentation of the left atrium, obtaining a single connected component solution, and separating the PVLI from the appendage).

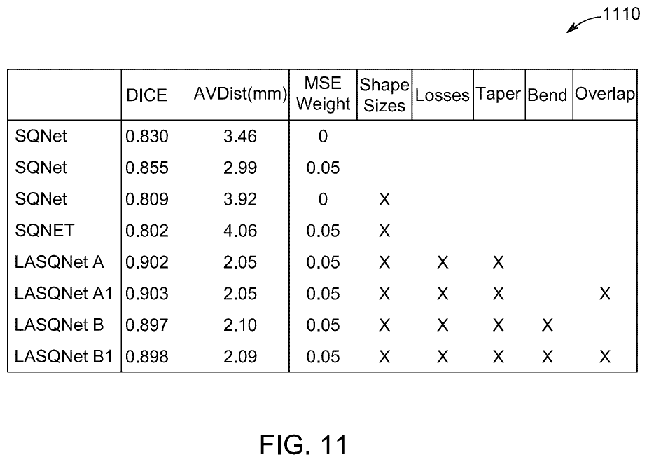

According to one or more embodiments, regarding a shape composition guidance and an initial testing phase, the mapping engine 101 seeks a solution that includes primitive shapes closely resembling the subject's various anatomical parts. To do this, the mapping engine 101 manipulates two parameters: the shape size transformation (which was controlled using the variables a for scale and b for bias) and wₘₛₑ, a weight applied to the mean shape loss that encouraged the network to find a solution with fewer shapes. Shape size transformation values can include for primitives 1-2, a=0.8, b=0.03; for primitives 3-4, a=0.5, b=0.02; and for the remaining primitives, a=0.02, b=0.01. In this regard, the mapping engine 101 performs a process of guiding the optimization to a solution made up of anatomical parts, as illustrated FIG. 10.

FIG. 10 depicts models 1010, 1020, 1030, and 1040 for shape composition guidance according to one or more embodiments, with original SQNet in top row (1010, 1020) and size guided on the bottom row (1030, 1040). Note that the anatomical parts include right superior (RS) in pink, right inferior (RI) in blue, left superior (LS) in green, left inferior (LI) in cyan, and appendage in yellow. Note that a solution with anatomically correct parts is found on the model 1040. The model 1010 provides an Original SQNet, wₘₛₑ=0. Model 1010 shows that without the mean shape loss, the network produced a mixture of shapes with no clear correspondence to specific anatomical parts. The model 1020 provides an Original SQNet, wₘₛₑ=0.05. Model 1020 illustrates that the mean shape loss alone was not sufficient to find an anatomical solution.

The model 1030 provides size guided, wₘₛₑ=0. As shown in model 1030, the mapping engine 101 was able to identify anatomically corresponding parts, along with many redundant primitives. The model 1040 provides size guided, wₘₛₑ=0.5. Model 1040 illustrates that when the mapping engine 101 applied the shape size guidance (by combining both methods), the mapping engine 101 was able to find an anatomical solution.

According to one or more embodiments, regarding LA reconstruction over synthetic paths, the mapping engine 101 used a dataset and evaluation metrics. For the dataset, experiments were conducted on a dataset of 1800 pairs of synthetic catheter paths and corresponding left atrial shapes. The left atrial shapes were generated, and the synthetic catheter paths were generated. These experiments allowed the mapping engine 101 to evaluate the performance of the proposed methods on a large and diverse dataset. For the evaluation metrics, the mapping engine 101 evaluated the performance of the networks. For instance, the mapping engine 101 used two metrics: DICE and average distance between boundary contours (AVDist=5 mm). DICE measures the similarity between the resulting volume and the ground truth shape, while the average distance between boundary contours compares the resulting boundary with the true boundary. The boundary is defined as the set of voxels separating the chamber's interior from the exterior. By using these two metrics, the mapping engine 101 is able to assess the overall accuracy of the network's reconstructions in terms of both the overall volume and the specific boundary contours. AVDist is described using Equation 15, where d(u, v) is the Euclidean distance between voxels u and v.

$$( \partial x , \partial y ) = 0.5 \frac{{\sum }_{u ∈ \partial x} min \{ d ( u , v ) : v ∈ \partial y \}}{‖ \partial x ‖} + 0.5 \frac{{\sum }_{u ∈ \partial y} min \{ d ( u , v ) : v ∈ \partial x \}}{‖ \partial y ‖} \qquad Equation 15$$

Both SQNet and LASQNet showed fast inference times, taking less than 5 ms to process 20 samples on a moderate consumer-grade Nvidia GTX2080 graphics card.

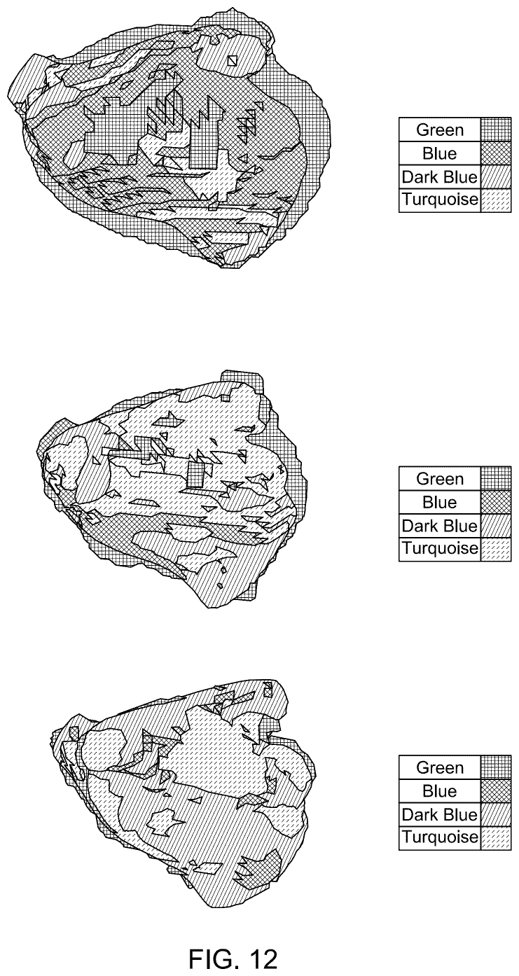

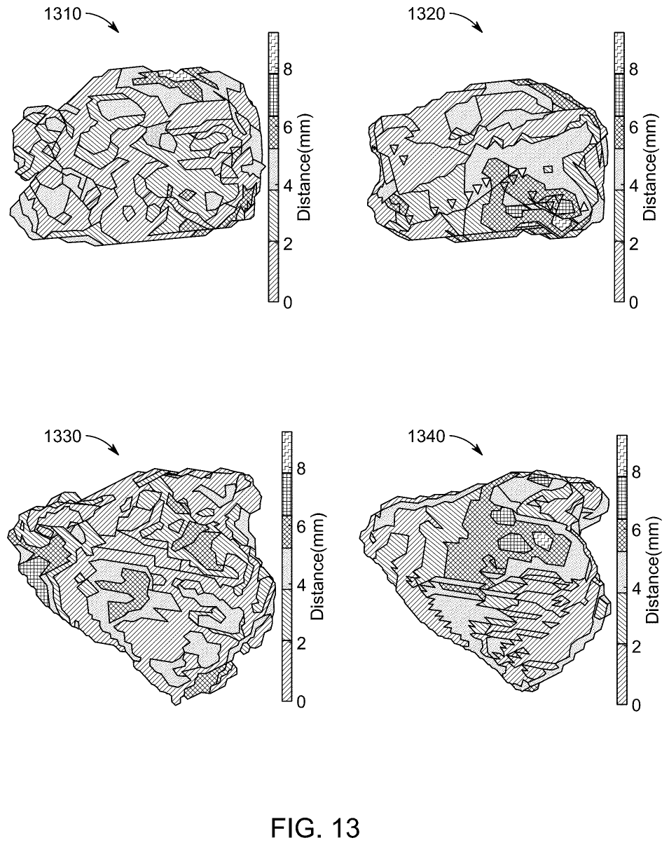

FIG. 11 depicts a table 1110 according to one or more embodiments. The table 1110 reports on the results of an ablation study of various variants of SQNet and LASQNet using different losses and transformations in continuous training runs A and B for LASQNet. Models 1210, 1220 and 1230 (in FIG. 12) demonstrates that, in all variants, SQNet tended to produce inflated solutions due to the low cost of the SQNet loss formulation for covering slightly extracardiac regions. Models 1210, 1220, and 1230 provide a reconstruction example over the test set. Blue is the ground truth, while reconstruction is in green. The reconstruction of SQNet is ‘inflated’ as compared to the ground truth, which did not occur for LASQNet reconstruction. The model 1210 provides guided SQNet, wmse=0.05. The model 1220 provides original SQNet, wmse=0.0. The model 1230 provides LASQNet B2. FWhile SQNet tended to produce inflated solutions due to the low cost of the SQNet loss formulation for covering slightly extracardiac regions, the use of additional losses, as seen in model 1230, helped to address this issue. The mapping engine 101 also found that the additional transformations had minimal impact on the quantitative measurement, as they primarily affected local structures.

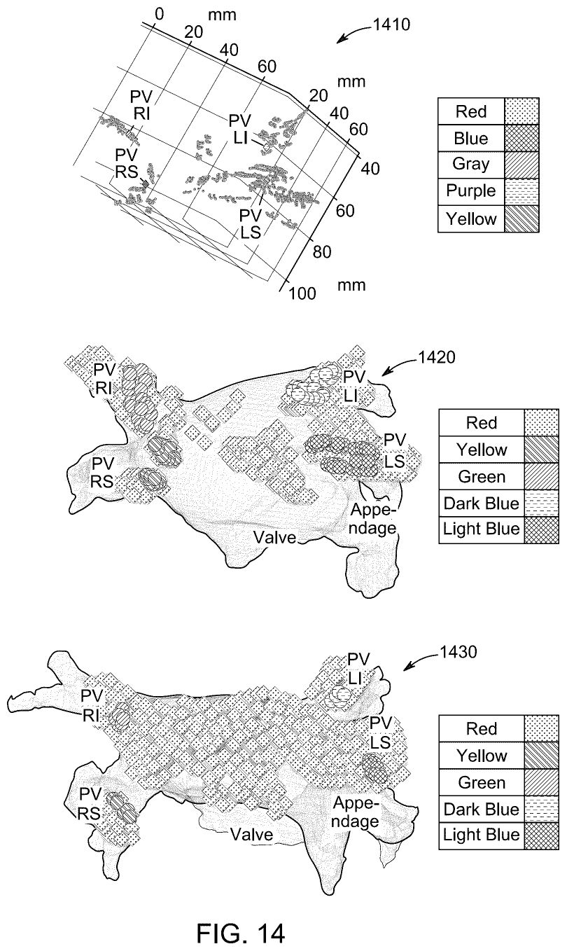

Although the LASQNets showed only a slight better quantitative results compared to the mean shape, a visual examination of the spatial distance error as shown in the models of FIG. 13 reveals that the error is greater for the mean shape solution at some of the pulmonary veins and their ostia, while the LASQNet has a more evenly distributed error over smaller regions due to having fewer degrees of freedom to describe the left atrial body. FIG. 13 depicts models 1310, 1320, 1330, and 1340 according to one or more embodiments. The models 1310, 1320, 1330, and 1340 provide a reconstruction colored by distance to the ground truth, compared to the mean shape solution over two views. The mean shape solution has greater errors over PVs and appendage. The model 1310 provides a top LASQNET B. The model 1320 provides a top mean LA. The model 1330 provides a side LASQNET B. The model 1340 provides a side mean LA.

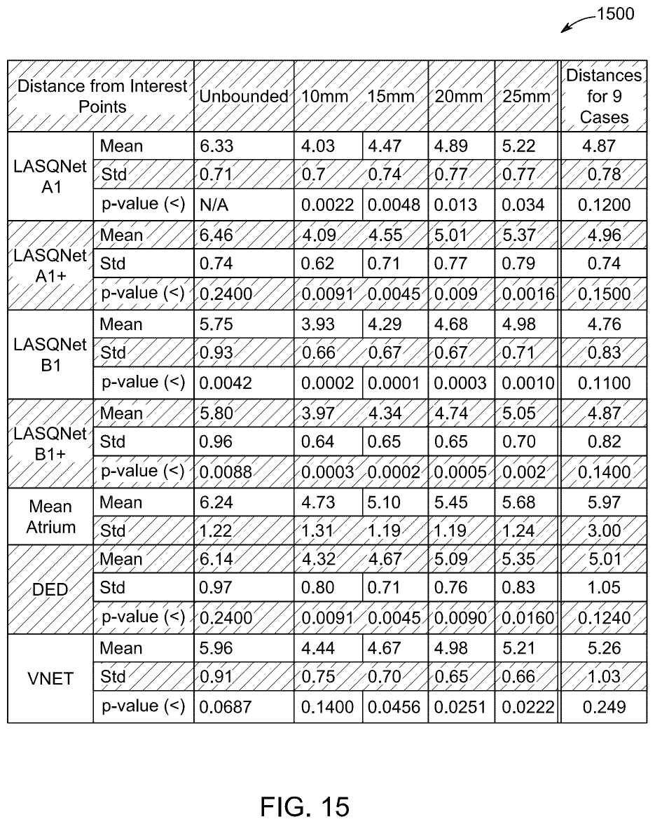

According to one or more embodiments, regarding LA reconstructions over human clinical cases, the mapping engine 101 performed data acquisition, an evaluation by contact points, and an evaluation with ground truth CT. For data acquisition, the mapping engine 101 used a dataset that consists of 80 clinical cases, where 26 of these had a properly registered mesh from a CT segmentation. The initial bearing path and tagged points belonging to each pulmonary vein (PV) ostia were acquired in under three minutes at the beginning of the procedure. The input point cloud was transformed and converted into an occupancy volume, which was the expected input for the network. The network output was converted into a triangular mesh using the marching cubes algorithm. FIG. 14 depicts graphs 1410, 1420, and 1430 from the clinical cases. The acquired path is in red, the synthetic template path is in blue, and the CT is in gray. Tagged PV points are color-coded. The graph 1410 (e.g., a focal catheter point cloud path) illustrates the input point cloud (path) with tagged PVs centroids. The graphs 1420 (e.g., a focal catheter voxelized path) and 1430 (e.g., a Lasso catheter voxelized path) show the input volumes in red, with colored tagged points for each PV (PVRS yellow, PVRI green, PVLI blue, PVLS light blue) and the registered CT. The performance of the reconstructions was evaluated using two methods: one using contact points IV-C.2 and the other comparing to ground truth CTs IV-C.3. The input point cloud and path may differ based on the type of catheter used, and there may be discontinuities in the acquired path due to suspension of position acquisition when the catheter exceeds a predefined velocity. The protocol may also deviate from the defined protocol and multiple arm catheters or different catheter maneuvers may be used. As explained below, performance of the LASQNet was compared to that of the conventional LA reconstruction methods, DED and V-Net for LA reconstruction.

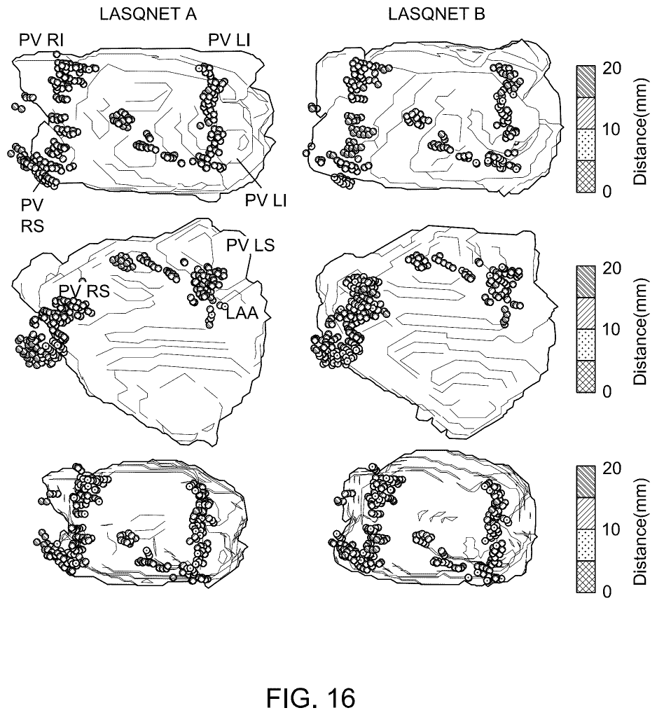

According to one or more embodiments, the mapping engine 101 provided evaluations by contact points. In this evaluation, the mapping engine 101 used a force-sensing catheter (THERMOCOOL SMARTTOUCH® SF Catheter) to acquire points on the surface during respiratory gating (end-expirium) with a contact force between 5 g and 15 g. These points are accurate (with an error of less than 1 mm) and often located near important anatomical landmarks and ablation regions. The study focused on cases that used a focal catheter and had a point cloud covering most of the synthetic path parts, resulting in a total of nine suitable cases. The reconstruction accuracy was measured as the mean distance between ground truth points and the nearest vertex on the reconstructed mesh. FIG. 15 depicts a table 1500 that provides clinical cases results. The left columns show surface to surface distances (mean and standard deviation) comparing LA reconstructions to ground truth CT over 26 clinical cases. Results are shown for LASQNets, DED, V-Net[ ], and the mean atrium, for unbounded distance and four different radii. The p-value tests for significant differences between the network and the mean shape. P-value under the significance level of 0.05 is in italics. The right column shows the distances to the contact points over the nine cases is inherently constrained to be a connected component. This effect was observed for the LA reconstruction task using all networks, including the DED and V-Net networks. The reconstruction accuracy results for the contact point evaluation are compared in the table 1500 (rightmost column). Thus, the table 1500 shows an advantage for the LASQNets, comparable results for the DED, while the V-Net lagged behind. All the results were highly better than the mean shape.

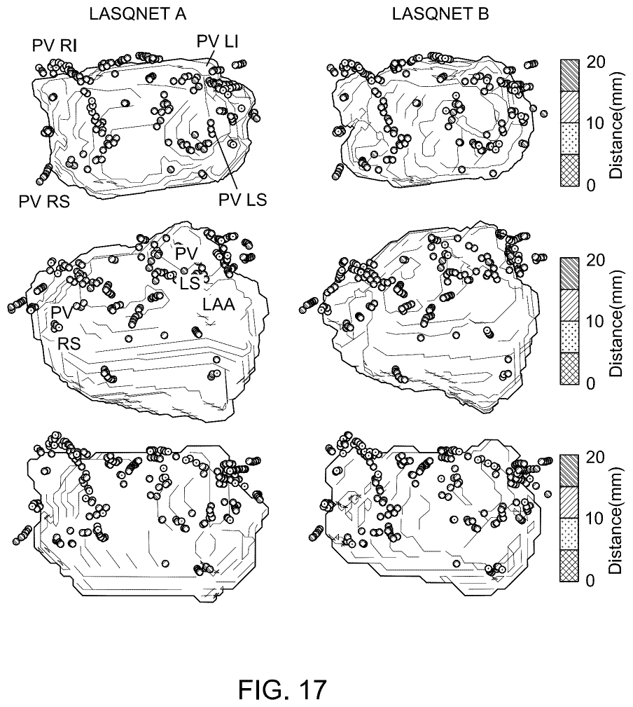

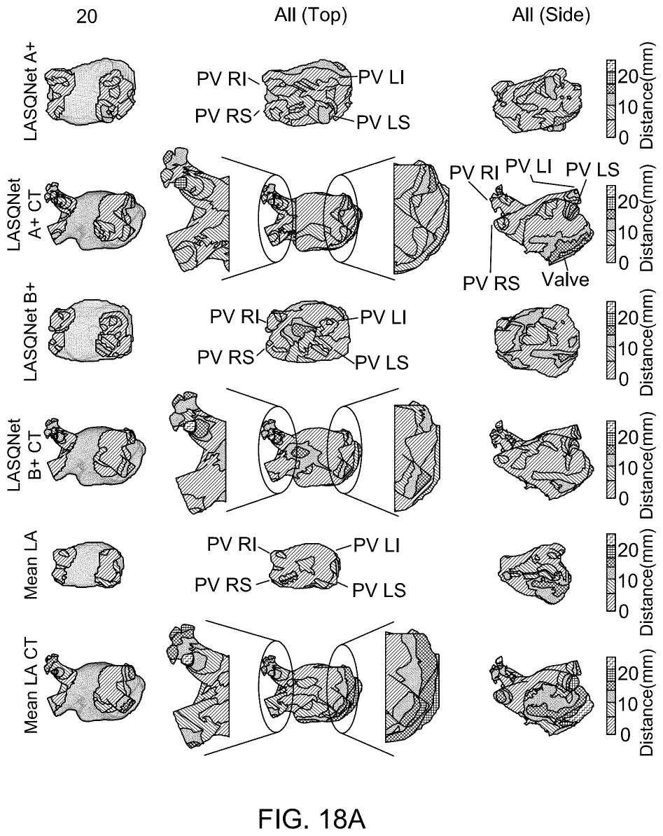

FIGS. 16 and 17 show two clinical cases reconstruction results for LASQNet A+ and LASQNet B+ networks. In each figure, the top row is a top-down view, the middle is a side of the PV's, and the bottom is a transparent reconstruction showing all ground truth points, color-coded by distance to reconstruction. In the figures, the ground truth points are overlaid and colored based on their distance to the reconstruction. The coloring of the ground truth points illustrates the distance between them and the provided reconstruction. The figures show that clinically important places such as PVs ostia and points around the ridges have low error values (typically less than 10 mm). The defined transformations allow the LASQNets to adapt the PV shapes better. The bending transformation effect can be seen in the results for LASQNet B+.

According to one or more embodiments, the mapping engine 101 provides evaluation with ground truth CT. For example, a dataset contains 26 cases with a CT registered to the acquired point cloud, which was used to align the CT with the network reconstruction. The mapping engine 101 compares the CT surfaces to the reconstructed mesh using surface-to-surface distances, focusing on clinically relevant regions. The surface-to-surface distance was measured by taking the nearest vertex on the second mesh and averaging the distance for all examined vertices. To focus on important regions, the mapping engine 101 only considered vertices within a defined radius of tagged PV points. A radius of 15 mm was found to capture the surroundings of the PV ostia well. In table 1500, the mapping engine 101 presents the quantitative results for all 26 CT cases. The reported p-value indicates the significance of the improvement of LASQNet B vs. the mean shape (paired t-test, one-tailed), and the improvement in comparison to V-Net and DED. For unbounded distance, the accuracy is improved by 0.4 mm, while for the clinically relevant regions, the improvement is in the range of 0.6-0.8 mm. Even though LASQNet-A lags in comparison to the mean shape in the unbounded metric, the accuracy is better than the mean shape in the clinically relevant regions and on par with the V-Net.

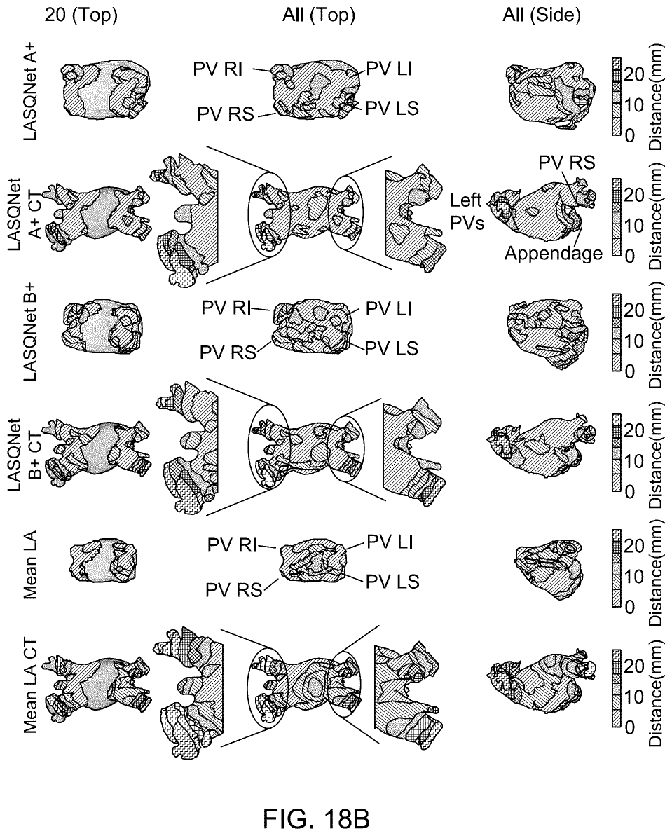

FIGS. 18A and 18B depict clinical cases according to one or more embodiments. For example, two clinical cases with CT reconstruction result for LASQNet A+ and for LASQNet B+ networks compared to the mean shape color-coded by distance to reconstruction. Further, the figures present reconstruction examples for the LASQNets. In the figures, an additional visualization that includes the side view of the reconstruction is provided in the third column. The error maps in the enlarged regions (PVs) are much lower for the LASQNets compared to the mean shape, as visualized by the shift towards the darker colors, especially near the ostia and the ridges between the PVs.

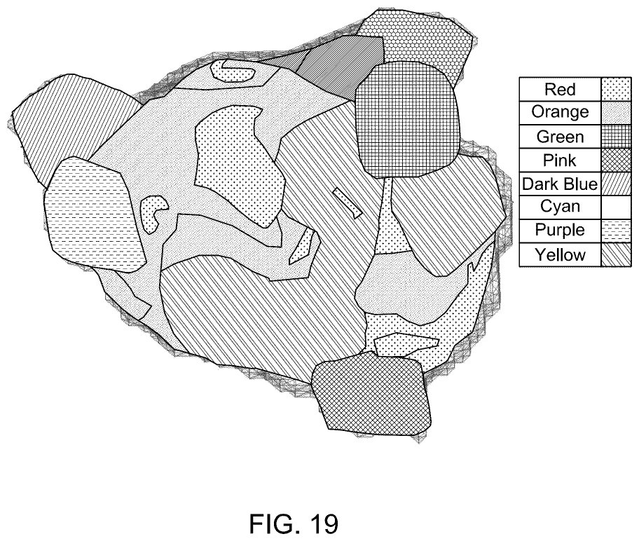

According to one or more embodiments, the mapping engine 101 provides utilization of the primitive representation framework. For instance, the mapping engine 101 demonstrates the utilization of primitive representations to add anatomical information and enforce anatomical constraints during the training process and after a result was obtained. A prominent task is segmenting the reconstruction into semantically meaningful anatomical parts. The LASQNet solution performs the anatomical parts segmentation as illustrated in FIG. 19. That is, FIG. 19 provides a LASQNET A+ clinical case reconstruction with anatomical segmentation. RS in pink, RI in blue, LS in green, LI in cyan, and appendage in yellow. The reminder composes the LA body and value. Each PV and appendage is represented using a single primitive, while the body and valve comprise the remainder of primitives.

Note that proper segmentation requires the result to be a connected component. Experiments that demonstrate the procedure to deal with non-connected results are described in the next section.

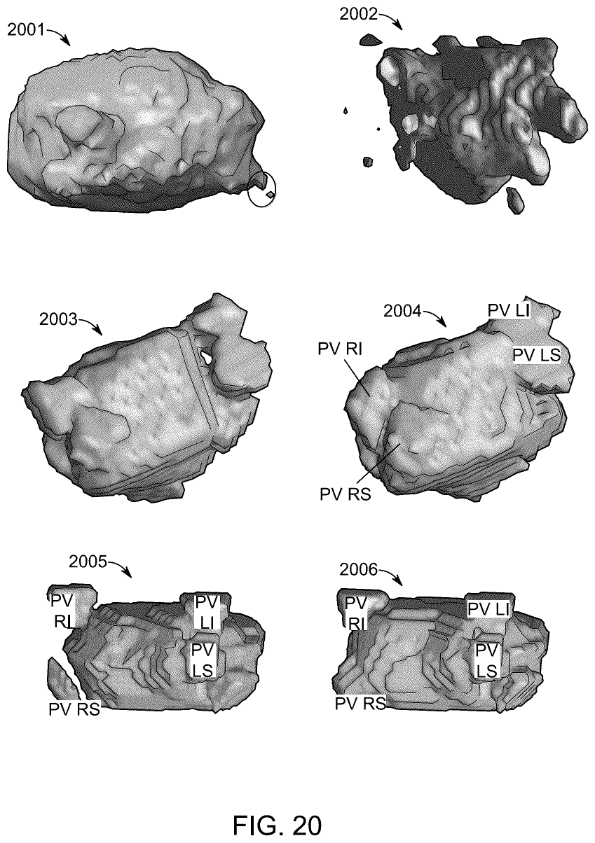

FIG. 20 depicts 3D reconstructions 2001, 2002, 2003, 2004, 2005, and 2006 according to one or more embodiments. The mapping engine 101 can generate each of the 3D reconstructions 2001, 2002, 2003, 2004, 2005, and 2006. Each initial LASQNet training is without using the loss as the primitives set has not converged yet. The 3D reconstruction 2003 shows a reconstruction example for LASQNet A at this stage, where a fragile connection between some of the PVs and the LA body can be observed. An adequately connected LA is seen in the 3D reconstruction 2004 after the network was trained.

Even after this training with the overlap loss, disconnected components might occur due to the softness of the constraint. For those cases, mapping engine 101 utilized the procedure described herein to remedy the reconstruction post inference. The mapping engine 101 employed three iterations of LBFGS (most converge after a single one) for the results of LASQNet A. A disconnected LASQNet A result is shown in the 3D reconstruction 2005, while the result after the correction (LASQNet A+) is depicted in the 3D reconstruction 2006.

As described herein, it is common for various reconstructions to eliminate the ridge between the PVLS and the appendage. By using the separation cost post inference optimization described herein, the system is able to reopen this ridge for a given LASQNETB reconstruction result

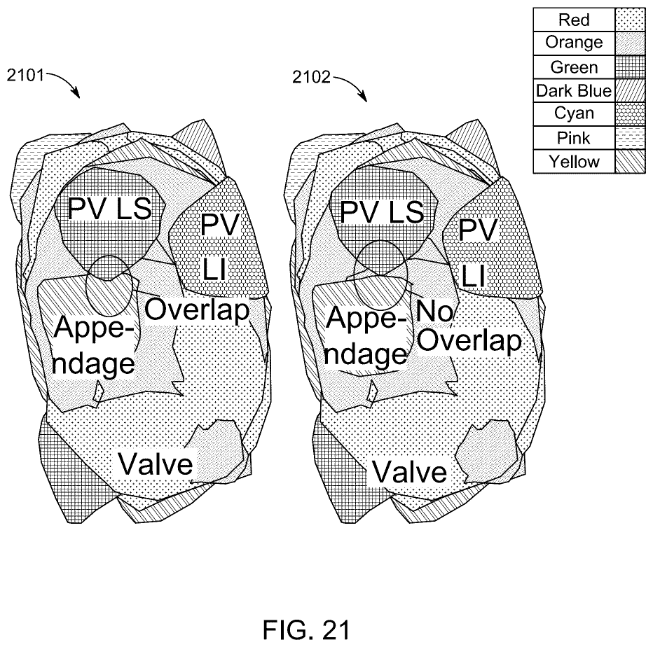

FIG. 21 depicts reconstructions 2101 and 2102 before and after applying the optimization according to one or more embodiments. As shown in the reconstruction 2101 (e.g., the original LASQNETB solution), the PV LS (green) and the appendage (yellow) overlap. As shown in the reconstruction 2102, by the mapping engine 101 using shape-apart optimization, the PV LS (green) and the appendage (yellow) are separated.

According to one or more embodiments, the mapping engine 101 can demonstrate how to optimize a primitive based neural-network in a controlled way to recover a shape and anatomical parts of the LA from catheter paths. Further, the mapping engine 101 (due to a robust build) provides proper reconstruction and segmentation to the required anatomical parts in human clinical cases.

According to one or more embodiments, the mapping engine 101 can guide the shapes size and number, such as in a semi-supervised manner, to reach an anatomically ‘correct’ solution (e.g., reconstruction). According to one or more embodiments, guiding the shapes size and number can be fully supervised if a target shape is fully modeled previously by the mapping engine 101 or the like.

Using the anatomically ‘correct’ solution, the mapping engine 101 defines a set of anatomical constraints and transformations. The mapping engine 101 shows this capability by example using the overlap constraint. As shown in FIG. 20, the 3D reconstructions 2001 and 2002 respectfully include, for example, DED and V-Net, which exhibit disconnected components and uses a LASQNet overlap constraint during the training phase of the mapping engine 101. The mapping engine 101 can utilize the same inputs to generate the 3D reconstructions 2003 without the constraint and the 3D reconstructions 2004 with the constraint. The mapping engine further generates a disconnected reconstruction, as shown in the 3D reconstruction 2005, and post-inference corrected reconstruction, as shown in the 3D reconstruction 2006.

In these test cases, shape guidance and the additional loss functions are used to achieve a proper solution using SQNets. These test cases have shown slight advantage for LASQNet over the mean shape solution in global metrics such as AVdist and DICE, and a disadvantage as compared to the DED solution TODO REF (i.e., because the LASQNet model has much fewer degrees of freedom as compared to the DED and less likely to obtain a fit in a globally over the shape). Degrees of freedom of the mapping engine 101 are utilized in a way that it can fit in clinically interesting places (e.g., around anatomical features and their properties), and which also show a better spatial error distribution as compared to the mean shape solution.

According to one or more embodiments, the mapping engine 101 can utilize additional features cost and constraints, such as the size and centers of ridges, constraint on PV directions, and so on. The features cost and constraints can be integrated both as a prior cost optimization and as a post inference optimization by the mapping engine 101. In turn, the mapping engine 101 can fix the model based on physician input that controls specific features. For example, the physician might deduce the location of a specific point that is the center of the ridge. If the mapping engine 101 defines a feature that finds the ridge center based on the anatomical parts, the mapping engine 101 can optimize the result to fit the point specified by the physician by using post inference optimization.

According to an exemplary embodiment, the mapping engine 101 can construct the one or more 3D models of an atrium with a trained neural network based on recorded trajectory of a catheter within the atrium. Further, the trained neural network can receive one or more inputs, which may include at least a dataset of three-dimensional (3D) atria shapes and corresponding catheter trajectories.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random-access memory (RAM), a register, cache memory, semiconductor memory devices, magnetic media such as internal hard disks and removable disks, magneto-optical media, and optical media such as CD-ROM disks, and digital versatile disks (DVDs).

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. A computer readable medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Examples of computer-readable media include electrical signals (transmitted over wired or wireless connections) and computer-readable storage media. Examples of computer-readable storage media include, but are not limited to, a register, cache memory, semiconductor memory devices, magnetic media such as internal hard disks and removable disks, magneto-optical media, optical media such as compact disks (CD) and digital versatile disks (DVDs), a random-access memory (RAM), a read-only memory (ROM), an erasable programmable read-only memory (EPROM or Flash memory), a static random-access memory (SRAM), and a memory stick. A processor in association with software may be used to implement a radio frequency transceiver for use in a terminal, base station, or any host computer.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one more other features, integers, steps, operations, element components, and/or groups thereof.

The descriptions of the various embodiments herein have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
acquiring one or more catheter trajectories in real-time during an ablation procedure;
training a pre-trained neural network based on a dataset and the one or more catheter trajectories to provide a trained neural network;
approximating an atrium shape utilizing the trained neural network and one or more portions of a catheter traversal path;
generating a three-dimensional model output from the trained neural network and the atrium shape; and
displaying the three-dimensional model output as an early visualization in the ablation procedure.

2. The method of claim 1, further comprising performing a pre-training of one or more neural networks to provide the pre-trained neural network.

3. The method of claim 2, wherein inputs for the pre-training comprise training one or more geometric primitives, and one or more trajectories.

4. The method of claim 2, wherein output primitives are modified by translation, rotation, size, shape, existence probability, tapering, and bending parameters.

5. The method of claim 1, wherein the one or more catheter trajectories comprise an input path representing an occupancy volume.

6. The method of claim 1, wherein the pre-trained neural network comprises a superquadratics neural network.

7. The method of claim 1, wherein the training is performed using one or more loss parts.

8. The method of claim 1, wherein the training is performed using a linearity transform.

9. The method of claim 8, wherein the linearity transform is configured to bias output of the neural network to produce geometry characteristic of a left atrium.

10. A non-transitory computer-readable medium storing instructions that, when executed by a processor, cause the processor to perform operations comprising:
acquiring one or more catheter trajectories in real-time during an ablation procedure;
training a pre-trained neural network based on a dataset and the one or more catheter trajectories to provide a trained neural network;
approximating an atrium shape utilizing the trained neural network and one or more portions of a catheter traversal path;
generating a three-dimensional model output from the trained neural network and the atrium shape; and
displaying the three-dimensional model output as an early visualization in the ablation procedure.

11. The non-transitory computer-readable of claim 10, wherein the operations further comprise performing a pre-training of one or more neural networks to provide the pre-trained neural network.

12. The non-transitory computer-readable medium of claim 11, wherein inputs for the pre-training comprise training one or more geometric primitives, and one or more trajectories.

13. The non-transitory computer-readable medium of claim 11, wherein output primitives are modified by translation, rotation, size, shape, existence probability, tapering, and bending parameters.

14. The non-transitory computer-readable medium of claim 10, wherein the one or more catheter trajectories comprise an input path representing an occupancy volume.

15. The non-transitory computer-readable medium of claim 10, wherein the pre-trained neural network comprises a superquadratics neural network.

16. The non-transitory computer-readable medium of claim 10, wherein the training is performed using one or more loss parts.

17. The non-transitory computer-readable medium of claim 10, wherein the training is performed using a linearity transform.

18. The non-transitory computer-readable medium of claim 17, wherein the linearity transform is configured to bias output of the neural network to produce geometry characteristic of a left atrium.

19. A system comprising:
a catheter configured to acquire one or more catheter trajectories in real-time during an ablation procedure; and
a processing system configured to:
train a pre-trained neural network based on a dataset and the one or more catheter trajectories to provide a trained neural network;
approximate an atrium shape utilizing the trained neural network and one or more portions of a catheter traversal path;
generate a three-dimensional model output from the trained neural network and the atrium shape; and
display the three-dimensional model output as an early visualization in the ablation procedure.

20. The system of claim 19, wherein the processing system further comprises a user interface for a user to edit a model based on input from a physician.
